(19) United States
(12) Patent Application Publication
(10) Pub. No.: US 2011/0282082 A1
(43) Pub. Date: Nov. 17, 2011

(54) METHOD FOR PRODUCING OXIDIZED COMPOUND

(75) Inventors: Tomonori Kawabata, Osaka (JP); Tetsuro Yonemoto, Osaka (JP)

(73) Assignee: Sumitomo Chemical Company, Limited

(21) Appl. No.: 13/119,161

(22) PCT Filed: Sep. 18, 2009
(86) PCT No.: PCT/JP2009/066851

§ 371 Date: May 26, 2011

(30) Foreign Application Priority Data
Sep. 19, 2008 (JP) 2008-240757
Dec. 12, 2008 (JP) 2008-316761

(51) Int. Cl. C07D 301/12; C01B 39/00

(52) U.S. Cl. 549/531; 423/700

(57) ABSTRACT

A method for producing an oxidized compound according to the present invention comprises reacting an organic compound with an oxidizing agent in the presence of titanosilicate (I) or a silylated form thereof, the titanosilicate (I) being obtained by contacting titanosilicate (II) with a structure-directing agent, and the titanosilicate (II) having an X-ray diffraction pattern reproduced in the form of interplanar spacings d of
1.24±0.08 nm,
1.08±0.03 nm,
0.9±0.03 nm,
0.6±0.03 nm,
0.39±0.01 nm and
0.34±0.01 nm.

### TECHNICAL FIELD

The present invention relates to a method for producing an oxidized compound.

### BACKGROUND ART

With respect to a method for producing an oxidized compound using a titanosilicate catalyst, Nonpatent Documents 1 and 2 disclose a method comprising epoxidizing cyclopentene through reaction with hydrogen peroxide in the presence of a Ti-MWW precursor catalyst, which catalyst is obtained by the acid treatment of a Ti-containing laminar compound with 2 M HNO₃. Patent Document 1 discloses a method for producing propylene oxide, comprising reacting propylene with hydrogen peroxide in the presence of the same catalyst as above.

Nonpatent Document 3 discloses a Ti-MWW precursor containing 13.5 wt % to 14.2 wt % of organic amine species, which is obtained by: mixing Ti-MWW, piperidine, and water; washing the obtained compound with water; and drying the compound overnight at 100° C. This Ti-MWW precursor has an Si/N ratio of 8.5 to 8.6 calculated from ICP (Si/Ti, Si/B) and CHN analyses described therein and thus has a higher nitrogen content than the Ti-MWW precursor described in Nonpatent Documents 1 and 2.

### CITATION LIST

[Nonpatent Document 1] Catalysis Today 117 (2006) 199-205
[Nonpatent Document 2] 91st CATSJ Meeting Abstracts: No. 1B07 (2003)
[Nonpatent Document 3] Journal of Physical Chemistry C, Vol. 112 No. 15, 2008
[Patent Document 1] Japanese Patent Laid-Open No. 2005-262164

### SUMMARY OF INVENTION

An object of the present application is to provide a novel method for producing an oxidized compound and titanosilicate.

Specifically, the present application relates to the following inventions.

[1] A method for producing an oxidized compound, comprising reacting an organic compound with an oxidizing agent in the presence of titanosilicate (I) or a silylated form thereof, the titanosilicate (I) being obtained by contacting titanosilicate (II) with a structure-directing agent, and the titanosilicate (II) having an X-ray diffraction pattern reproduced in the form of interplanar spacings d of
1.24±0.08 nm,
1.08±0.03 nm,
0.9±0.03 nm,
0.6±0.03 nm,
0.39±0.01 nm and
0.34±0.01 nm.
   [2] The method for producing an oxidized compound according to [1], wherein the organic compound is an olefin compound or an aromatic compound.
   [3] The method for producing an oxidized compound according to [1] or [2], wherein the titanosilicate (I) has a molar ratio of silicon to nitrogen (Si/N ratio) of from 5 to 20 inclusive.
   [4] The method for producing an oxidized compound according to any of [1]-[3], wherein the titanosilicate (I) has a ratio of a specific surface area (SH₂O) to a specific surface area (SN₂)(SH₂O/SN₂) of from 0.7 to 1.5 inclusive, the specific surface areas SH₂O and SN₂ being measured by water vapor adsorption and nitrogen adsorption methods, respectively.
   [5] The method for producing an oxidized compound according to any of [1]-[4], wherein the titanosilicate (II) is crystalline titanosilicate having an MWW or MSE structure, or a Ti-MWW precursor (a).
   [6] The method for producing an oxidized compound according to any of [1]-[5], wherein the structure-directing agent is piperidine or hexamethyleneimine, or a mixture thereof.
   [7] The method for producing an oxidized compound according to any of [1]-[6], wherein the contact of the titanosilicate (II) with the structure-directing agent is performed at a temperature from 0 to 250° C. inclusive.
   [8] Titanosilicate or a silylated form thereof, wherein the titanosilicate has a molar ratio of silicon to nitrogen (Si/N ratio) of from 10 to 20 inclusive.
   [9] The titanosilicate or a silylated form thereof according to [8], the titanosilicate being obtained by contacting titanosilicate (II) with a structure-directing agent, and the titanosilicate (II) having an X-ray diffraction pattern reproduced in the form of interplanar spacings d of
1.24±0.08 nm,
1.08±0.03 nm,
0.9±0.03 nm,
0.6±0.03 nm,
0.39±0.01 nm and
0.34±0.01 nm.
   [10] The titanosilicate or a silylated form thereof according to [9], wherein the titanosilicate (II) is crystalline titanosilicate having an MWW or MSE structure, or a Ti-MWW precursor (a).
   [11] Use of titanosilicate or a silylated form thereof according to any of [8]-[10] as a catalyst in a method for producing an oxidized compound.
   [12] A catalyst for oxidation reaction of an organic compound, comprising titanosilicate (I) or a silylated form thereof, the titanosilicate (I) being obtained by contacting titanosilicate (II) with a structure-directing agent, and the titanosilicate (II) having an X-ray diffraction pattern reproduced in the form of interplanar spacings d of
1.24±0.08 nm,
1.08±0.03 nm,
0.9±0.03 nm,
0.6±0.03 nm,
0.39±0.01 nm and
0.34±0.01 nm.
   [13] The method for producing an oxidized compound according to any of [1]-[7], wherein the oxidizing agent is oxygen or peroxide.
   [14] The method for producing an oxidized compound according to [13], wherein the peroxide is at least one compound selected from the group consisting of hydrogen peroxide, t-butyl hydroperoxide, t-amyl hydroperoxide, cumene hydroperoxide, methylcyclohexyl hydroperoxide, tetralin hydroperoxide, isobutylbenzene hydroperoxide, ethylnaphthalene hydroperoxide, and peracetic acid.
   [15] The method for producing an oxidized compound according to any of [1]-[7], [13] and [14], wherein the reaction is epoxidation reaction of an olefin compound or hydroxylation reaction of benzene or a phenol compound.
   [16] The method for producing an oxidized compound according to any of [1]-[7], [13], [14] and [15], wherein the reaction is epoxidation reaction of an olefin compound, and the oxidizing agent is hydrogen peroxide.
   [17] The method for producing an oxidized compound according to [16], wherein the oxidizing agent is hydrogen peroxide synthesized in the same reaction system as that of the epoxidation of an olefin compound.
   [18] The method for producing an oxidized compound according to any of [1]-[7], [13], [14], [15], [16] and [17], wherein the reaction is performed in the presence of an organic solvent selected from the group consisting of alcohol, ketone, nitrile, ether, aliphatic hydrocarbon, aromatic hydrocarbon, halogenated hydrocarbon, ester and mixtures thereof.
   [19] The method for producing an oxidized compound according to [18], wherein the organic solvent is acetonitrile or t-butanol.

The production method of the present invention is useful as a method for producing an oxidized compound. The titanosilicate (I) is useful as a catalyst for oxidation reaction of an organic compound.

### BRIEF DESCRIPTION OF DRAWINGS

FIG. 1 is a graph showing an X-ray diffraction pattern of catalyst A;

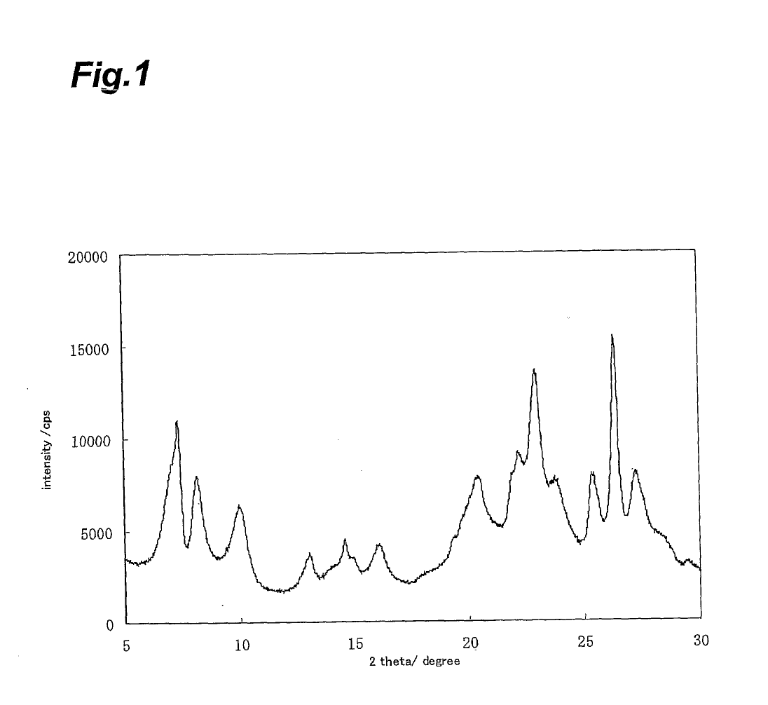

FIG. 2 is a graph showing an X-ray diffraction pattern of catalyst B;

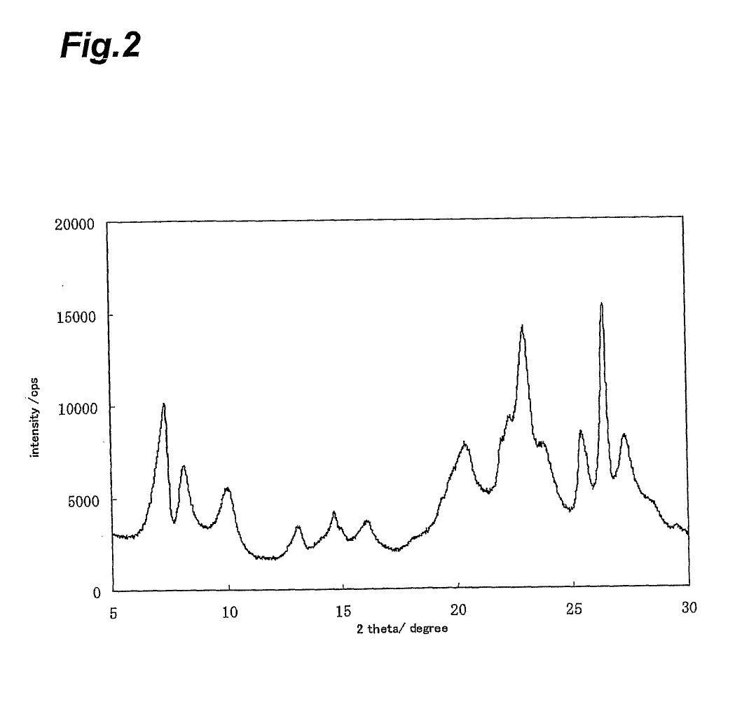

FIG. 3 is a graph showing an X-ray diffraction pattern of catalyst C;

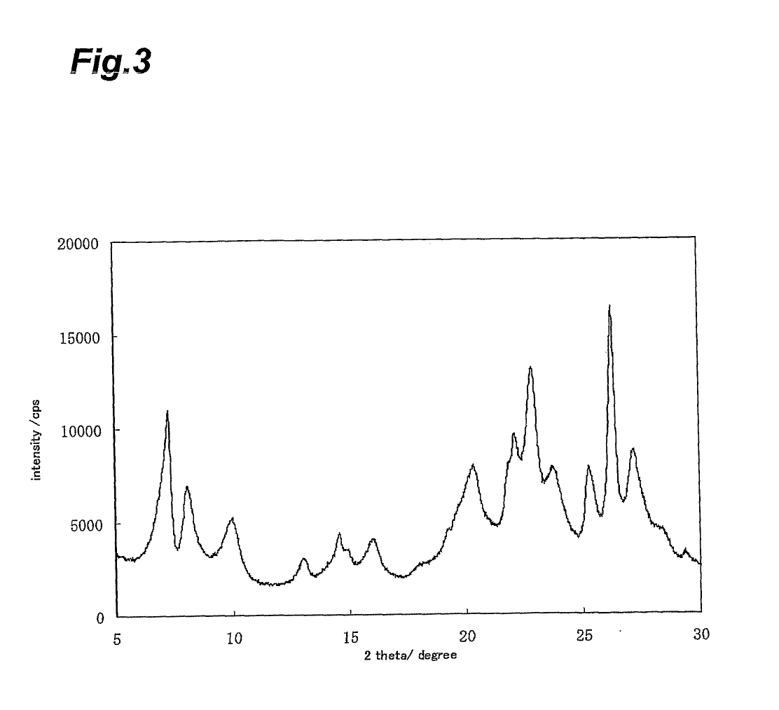

FIG. 4 is a graph showing an X-ray diffraction pattern of catalyst D;

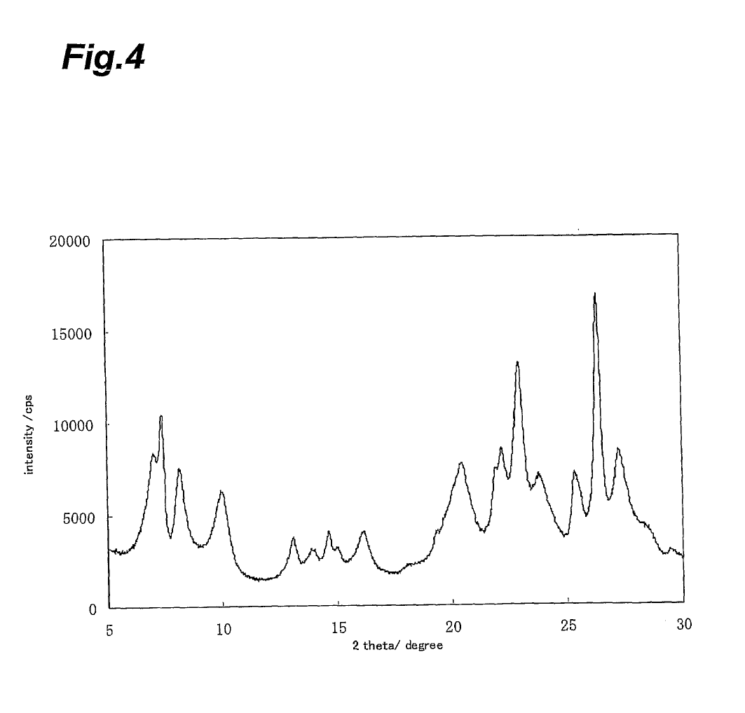

FIG. 5 is a graph showing an X-ray diffraction pattern of catalyst E;

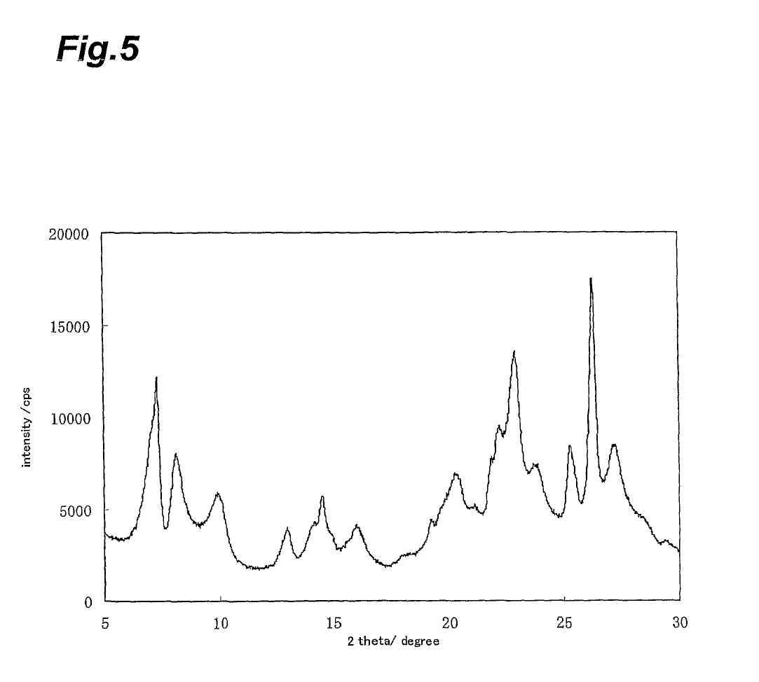

FIG. 6 is a graph showing an X-ray diffraction pattern of catalyst F;

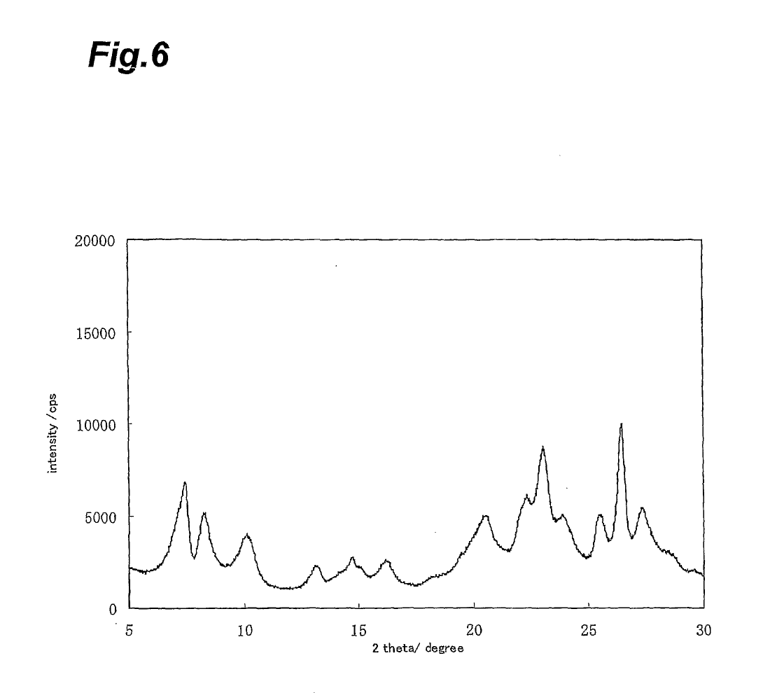

FIG. 7 is a graph showing an X-ray diffraction pattern of catalyst G;

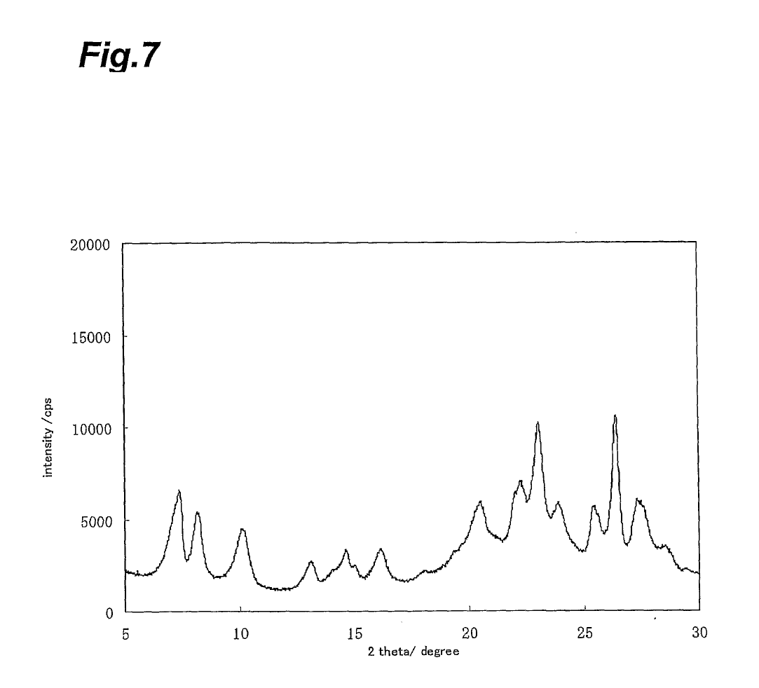

FIG. 8 is a graph showing an X-ray diffraction pattern of catalyst H;

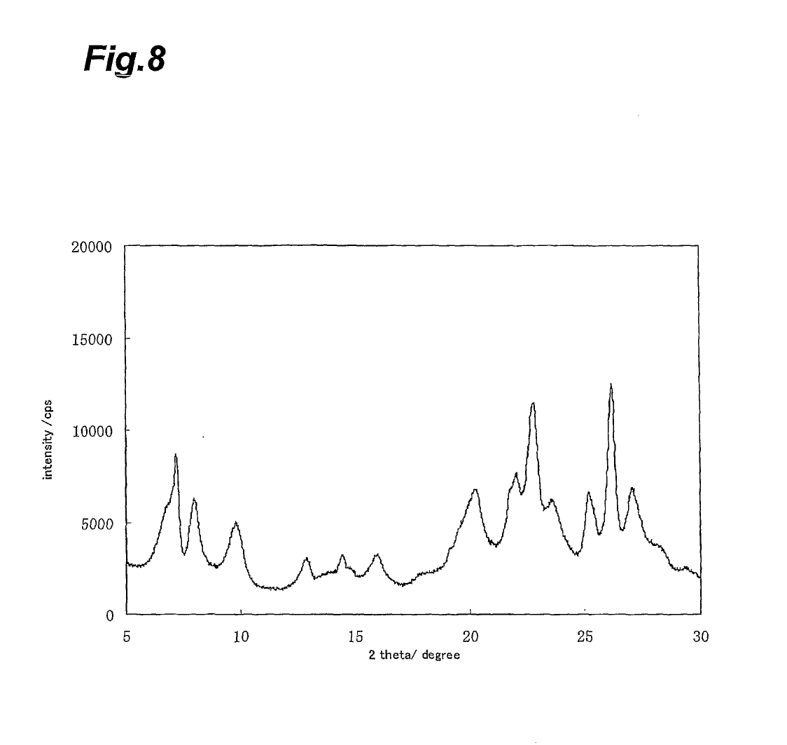

FIG. 9 is a graph showing an X-ray diffraction pattern of catalyst I;

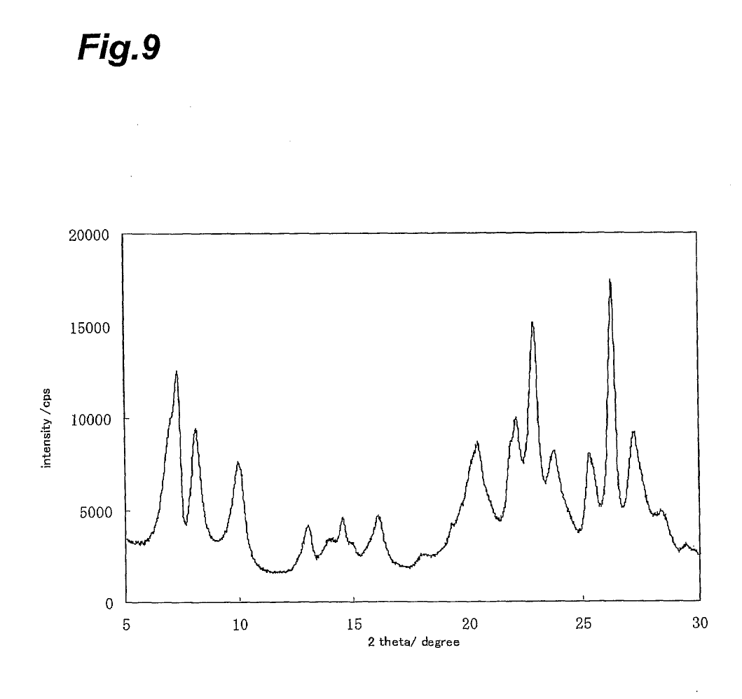

FIG. 10 is a graph showing an X-ray diffraction pattern of catalyst J;

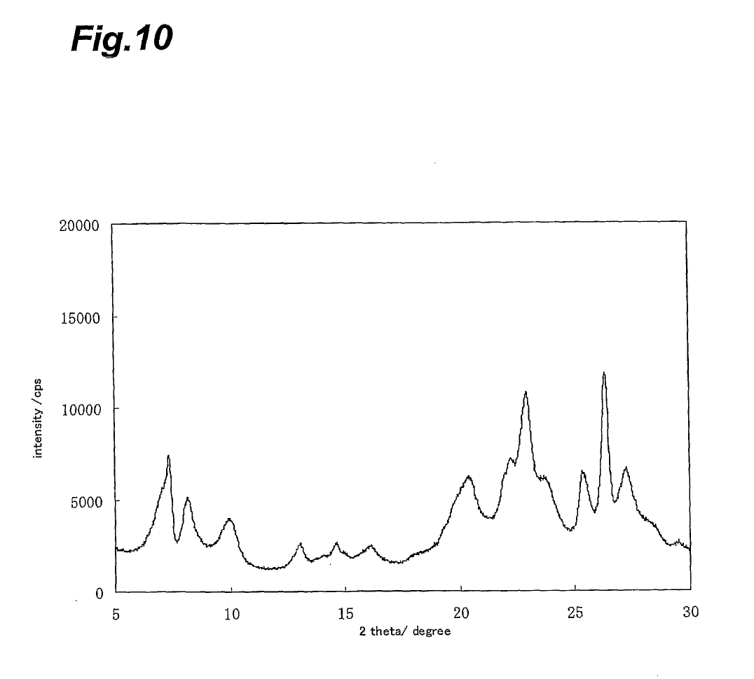

FIG. 11 is a graph showing an X-ray diffraction pattern of catalyst K;

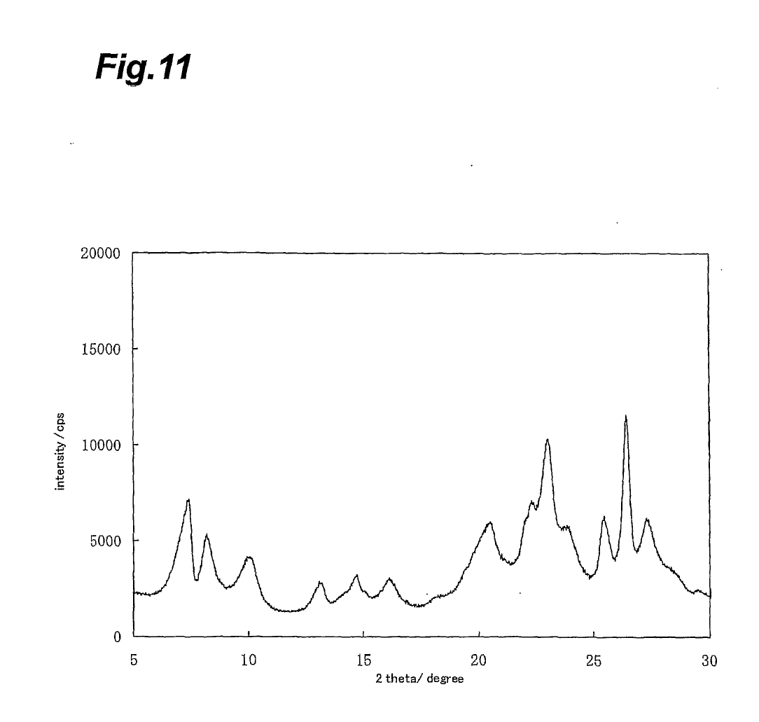

FIG. 12 is a graph showing an X-ray diffraction pattern of catalyst L;

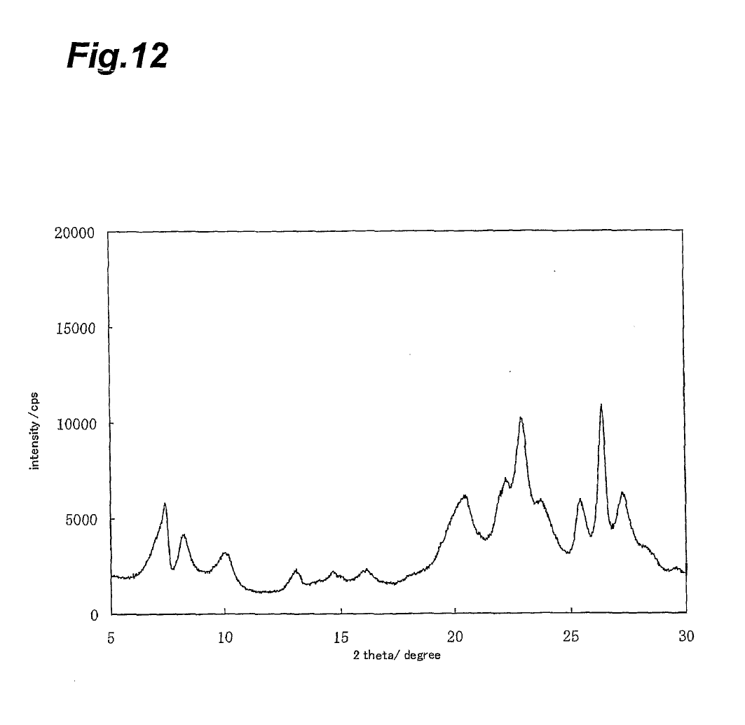

FIG. 13 is a graph showing an X-ray diffraction pattern of catalyst M;

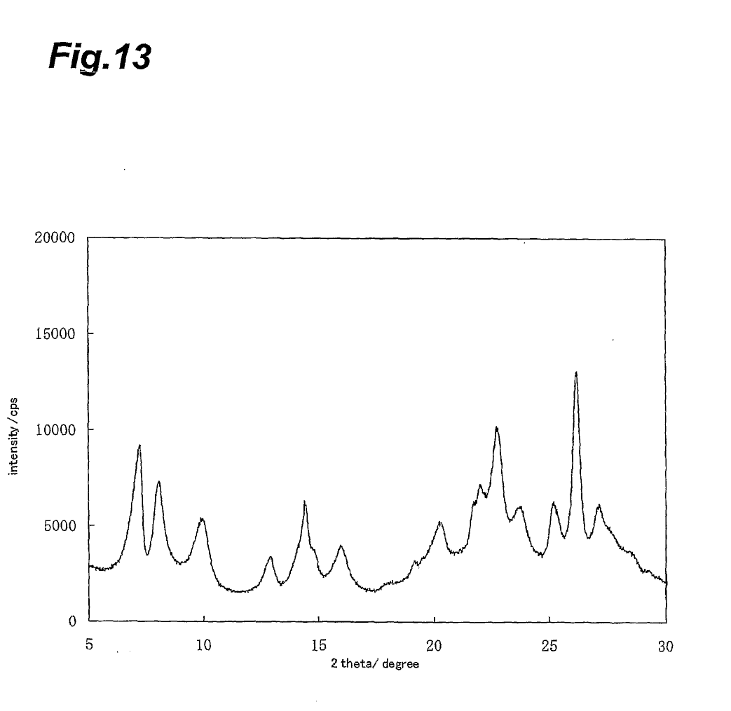

FIG. 14 is a graph showing an X-ray diffraction pattern of solid product 1;

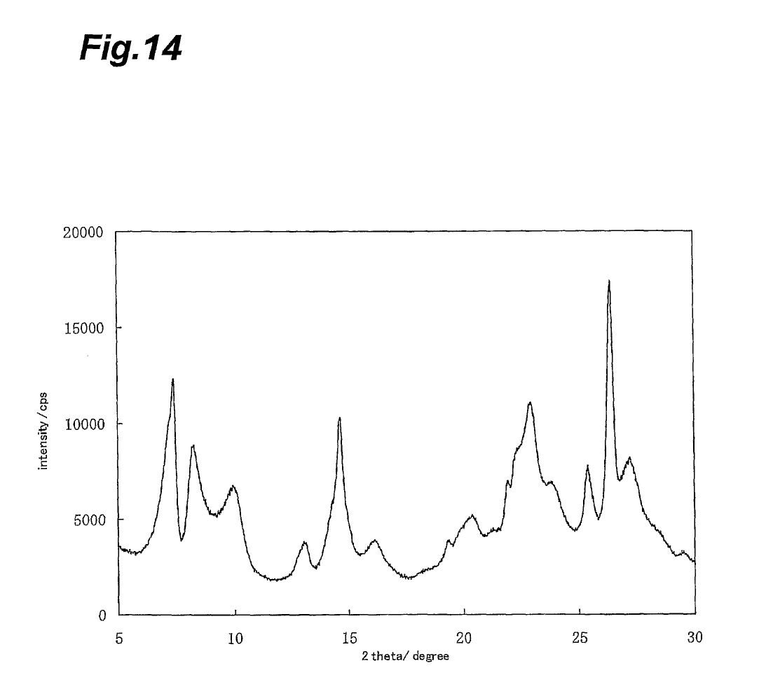

FIG. 15 is a graph showing an X-ray diffraction pattern of solid product 2;

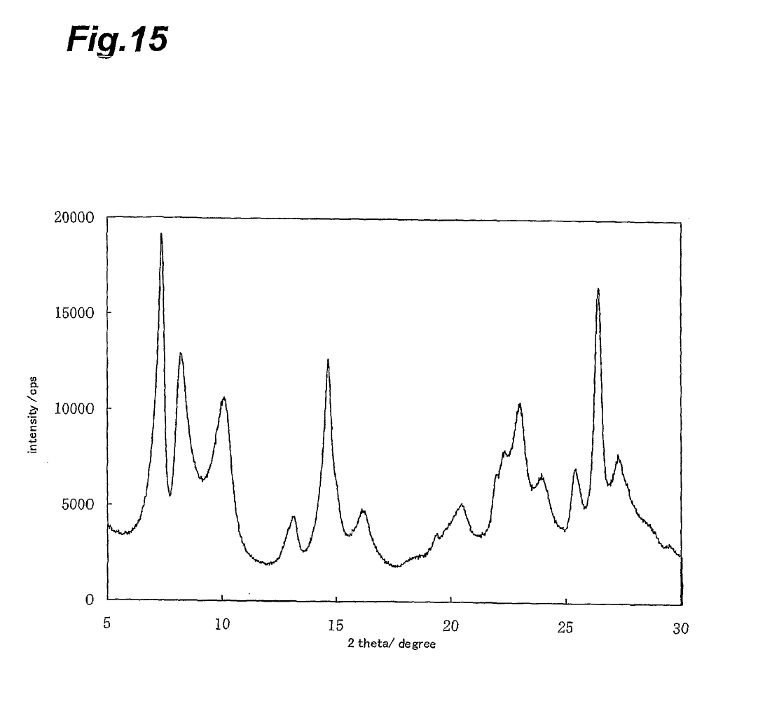

FIG. 16 is a graph showing an X-ray diffraction pattern of solid product 3;

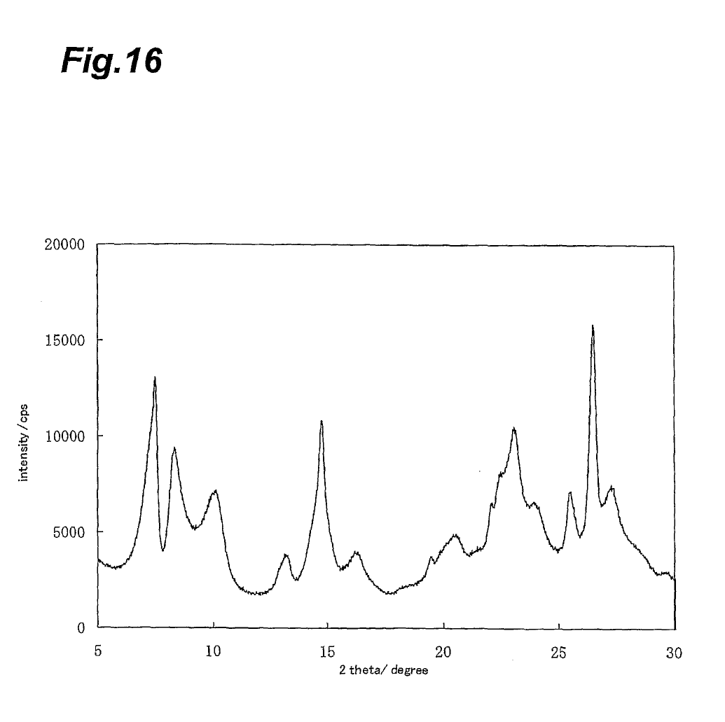

FIG. 17 is a graph showing an X-ray diffraction pattern of solid product 4;

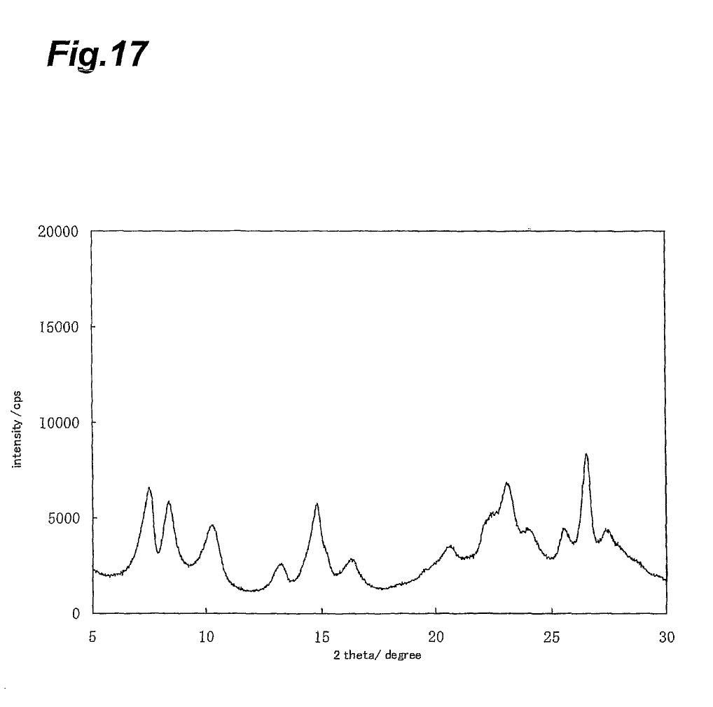

FIG. 18 is a graph showing an X-ray diffraction pattern of powder b3;

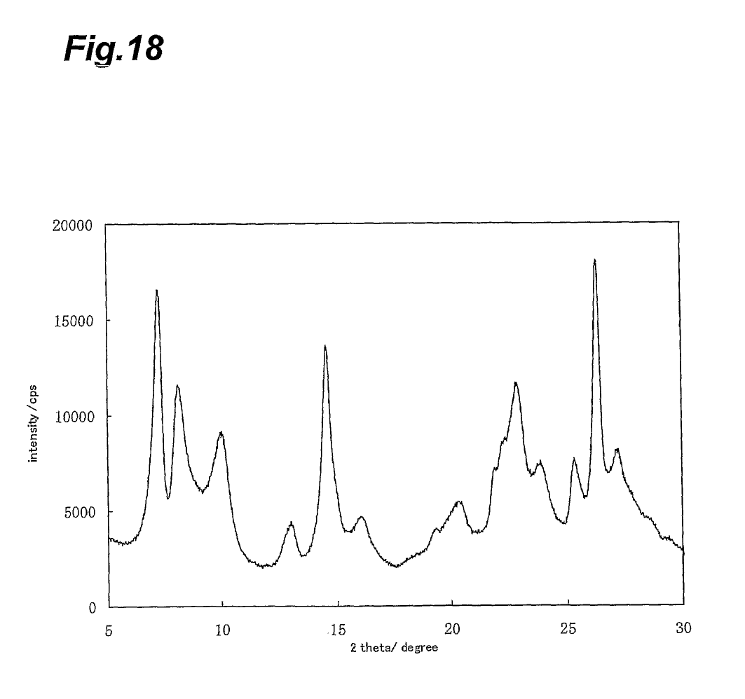

FIG. 19 is a graph showing an X-ray diffraction pattern of powder f2;

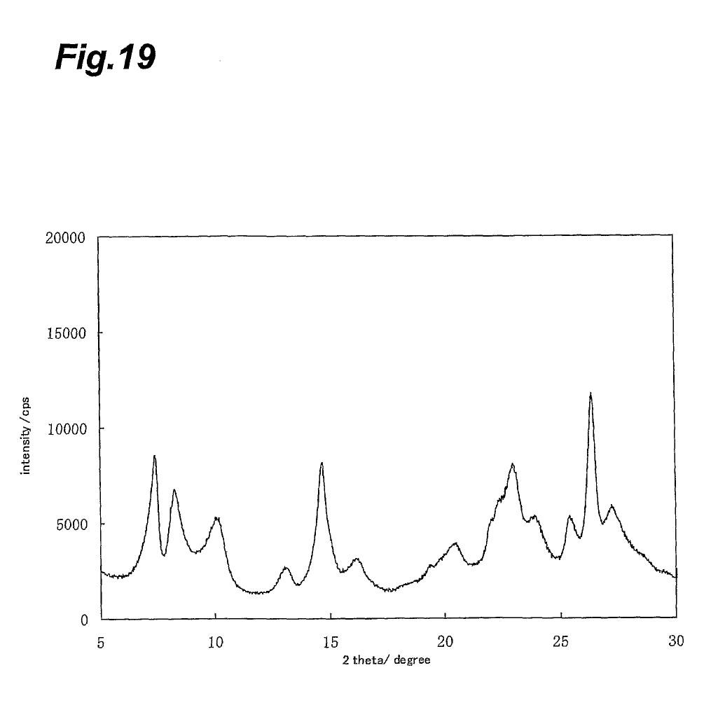

FIG. 20 is a graph showing an X-ray diffraction pattern of solid product g6;

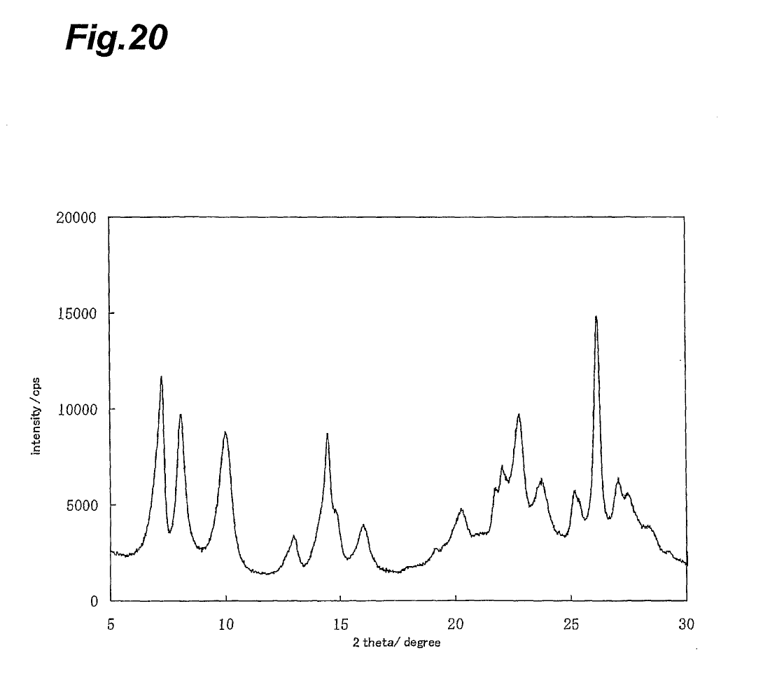

FIG. 21 is a graph showing an X-ray diffraction pattern of solid product h3;

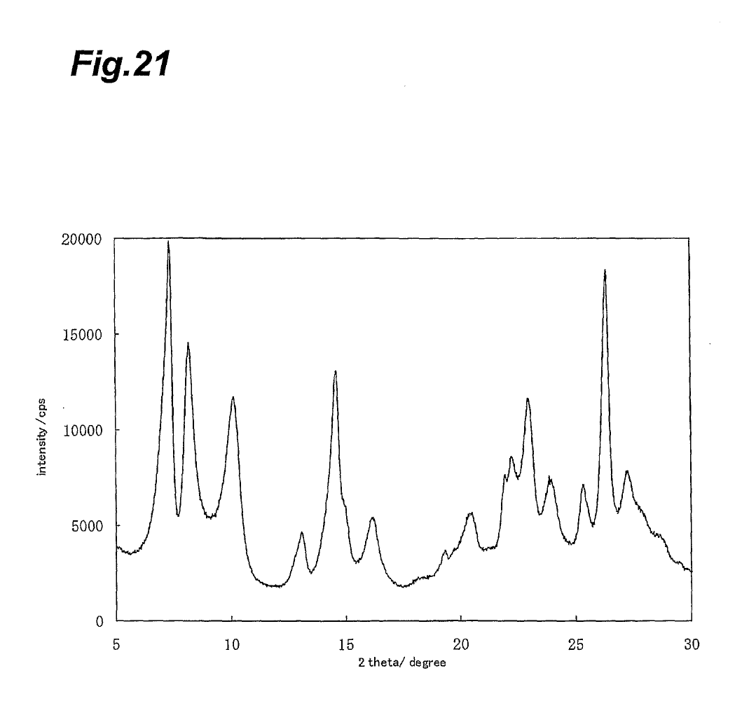

FIG. 22 is a graph showing an X-ray diffraction pattern of solid product i3; and

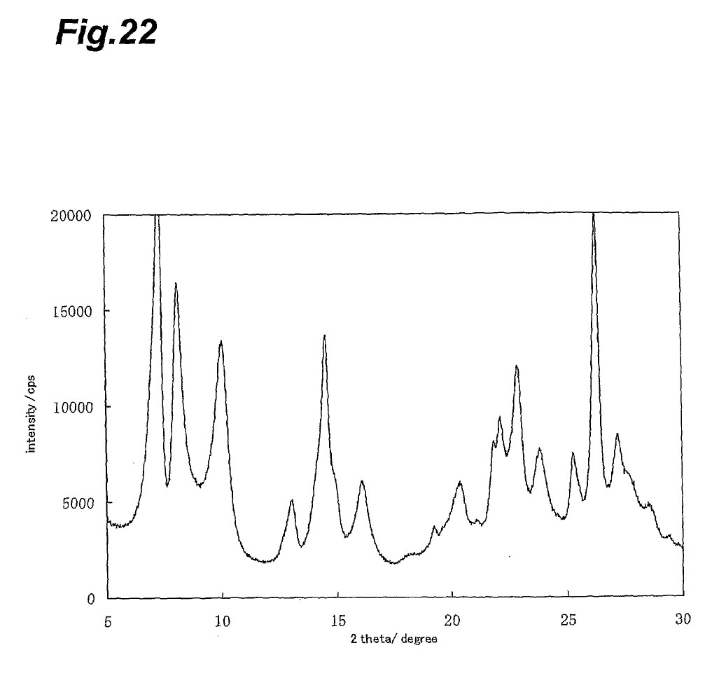

FIG. 23 is a graph showing an X-ray diffraction pattern of powder j2.

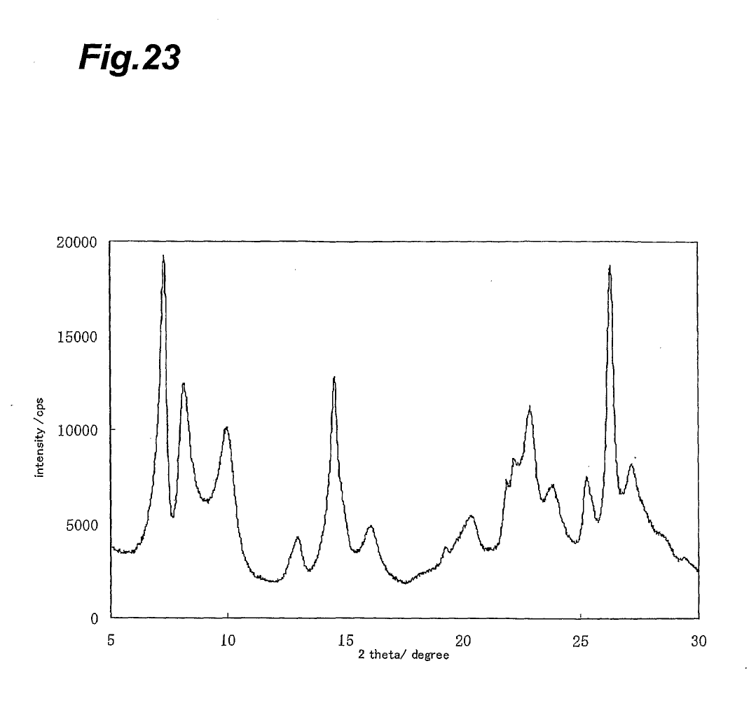

FIG. 24 is a graph showing an X-ray diffraction pattern of powder n2.

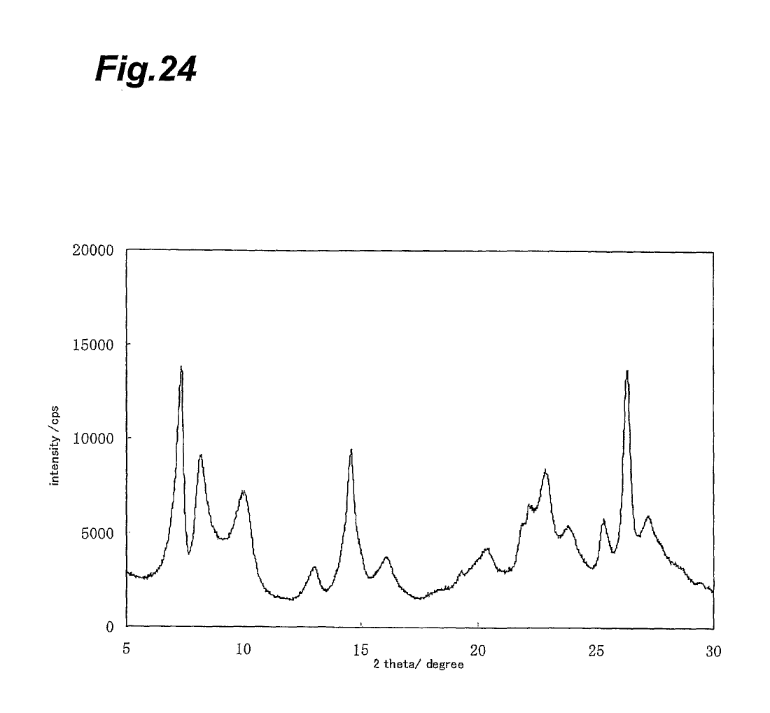

### DESCRIPTION OF EMBODIMENTS

A method for producing an oxidized compound according to the present invention comprises reacting an organic compound with an oxidizing agent in the presence of titanosilicate (I) or a silylated form thereof, the titanosilicate (I) being obtained by contacting titanosilicate (II) with a structure-directing agent, and the titanosilicate (II) having an X-ray diffraction pattern reproduced in the form of interplanar spacings d of
1.24±0.08 nm,
1.08±0.03 nm,
0.9±0.03 nm,
0.6±0.03 nm,
0.39±0.01 nm and
0.34±0.01 nm.

Titanosilicate is a generic name for silicate having tetracoordinated Ti. Titanosilicate herein can be confirmed that an UV-visible absorption spectrum of a wavelength region of 200 nm to 500 nm has the greatest absorption peak in a wavelength region of 220±10 nm (see e.g., Chemical Communications 1026-1027, (2002)). The UV-visible absorption spectrum can be measured by a diffuse reflection method using an UV-visible spectrophotometer equipped with a diffuse reflection attachment.

Ti-MWW means crystalline titanosilicate having an MWW structure. The MWW structure is a structure of a molecular sieve represented by the structural code specified by the International Zeolite Association (IZA). This structure has supercages (0.7×0.7×1.8 nm) having pores composed of an oxygen 10-membered ring and openings composed of an oxygen 10-membered ring and hemispherical side pockets having openings composed of an oxygen 12-membered ring.

The titanosilicate (I) is obtained by contacting titanosilicate (II) with a structure-directing agent and therefore presumably has, at a certain rate, pores containing the structure-directing agent in its porous structure derived form the titanosilicate (II). Such a porous structure as the titanosilicate (I) is confirmed from the X-ray diffraction pattern described later.

Furthermore, the titanosilicate (I) is obtained by the contact of the titanosilicate (II) with the structure-directing agent without being subjected to a calcination step and therefore differs in the X-ray diffraction pattern from the MWW structure, as described later. The titanosilicate (I) shows excellent activities as a catalyst for oxidation reaction of an organic compound.

The titanosilicate (I) exhibits an absorption peak in a wavelength region of 210 nm to 230 nm in an UV-visible absorption spectrum measured by a diffuse reflection method (standard for baseline: Spectralon) using an UV-visible spectrophotometer.

The titanosilicate (I) generally exhibits the following X-ray diffraction pattern:

Interplanar spacing d
1.24±0.08 nm (12.4±0.8 Å)
1.08±0.03 nm (10.8±0.3 Å)
0.9±0.03 nm (9±0.3 Å)
0.6±0.03 nm (6±0.3 Å)
0.39±0.01 nm (3.9±0.1 Å)
0.34±0.01 nm (3.4±0.1 Å)

The titanosilicate (I) further exhibits the relationship: an intensity ratio X¹/X² (X¹/X²=ratio of peak intensity X¹ at the interplanar spacing 9±0.3 Å to peak intensity X² at the interplanar spacing 3.4±0.1 Å) of larger than 0 and 0.4 or smaller, preferably not smaller than 0.05 and 0.4 or smaller in the X-ray diffraction pattern.

In the present specification, the X-ray diffraction pattern can be measured by irradiation with copper Kα X-rays using an X-ray diffractometer.

The titanosilicate (I) preferably has a molar ratio of silicon to nitrogen (Si/N ratio) of, but not particularly limited to, from 5 to 20 inclusive.

The Si/N ratio is more preferably 8, even more preferably 10, as the lower limit and is more preferably 35, even more preferably 18, particularly preferably 16, as the upper limit.

The titanosilicate (I) having an Si/N ratio within this range can show more excellent catalytic activity. One aspect of the present invention encompasses titanosilicate or a silylated form thereof, wherein the titanosilicate has a molar ratio of silicon to nitrogen (Si/N ratio) of from 10 to 20 inclusive.

The titanosilicate of the present invention and a silylated form thereof can respectively be prepared by the same method as that for the titanosilicate (I) and a silylated form thereof.

The molar ratio of silicon to nitrogen (Si/N ratio) is determined by subjecting a sample to elementary analysis. The elementary analysis can be conducted by a general method as follows: Ti (titanium), Si (silicon), and B (boron) can be measured by alkali fusion, dissolution in nitric acid, and ICP emission spectroscopy; and N (nitrogen) can be measured by oxygen circulating combustion and TCD detection systems.

The titanosilicate (I) usually has a ratio of a specific surface area (SH₂O) to a specific surface area (SN₂)(SH₂O/SN₂) of 0.7 or larger, preferably 0.8 or larger. The ratio SH₂O/SN₂ is usually 1.5, preferably 1.3, as the upper limit.

In the present invention, the specific surface area SN₂ is determined by the steps of degassing a sample at 150° C. and measuring the degassed sample by a nitrogen adsorption method, which area is calculated by a BET method. The specific surface area SH₂O is determined by the steps of degassing a sample at 150° C. and measuring the degassed sample at an adsorption temperature of 298 K by a water vapor adsorption method, which area is calculated by a BET method.

The titanosilicate (I) is obtained by the contact of the titanosilicate (II) with the structure-directing agent.

The silylated form of the titanosilicate (I) is obtained by silylating the titanosilicate (I) with a silylating agent, for example, 1,1,1,3,3,3-hexamethyldisilazane.

In the present specification, the structure-directing agent means an organic compound used for the formation of a zeolite structure. The structure-directing agent can form a precursor of the zeolite structure by organizing polysilicic acid or polymetasilicic acid ions into a topology around it (see Science and Engineering of Zeolite, pp. 33-34, 2000, Kodansha Scientific Ltd).

Any nitrogen-containing compound that can form zeolite having an MWW structure can be used as the structure-directing agent without particular limitations. Examples of the structure-directing agent include: organic amines such as piperidine and hexamethyleneimine; and quaternary ammonium salts such as N,N,N-trimethyl-1-adamantanammonium salts (N,N,N-trimethyl-1-adamantanammonium hydroxide, N,N,N-trimethyl-1-adamantanammonium iodide, etc.) and octyltrimethylammonium salts described in Chemistry Letters 916-917 (2007) (octyltrimethylammonium hydroxide, octyltrimethylammonium bromide, etc.). These compounds may be used alone or as a mixture of two or more thereof at an arbitrary ratio.

The structure-directing agent is preferably piperidine or hexamethyleneimine.

In the production of the titanosilicate (I), the structure-directing agent is usually used in an amount of 0.01 parts by weight, preferably 0.1 parts by weight, more preferably 1 part by weight, even more preferably 2 parts by weight, as the lower limit with respect to 1 part by weight of the titanosilicate (II) and in an amount of 100 parts by weight, preferably 50 parts by weight, more preferably 20 parts by weight, even more preferably 15 parts by weight, particularly preferably 10 parts by weight as the upper limit with respect to 1 part by weight of the titanosilicate (II).

By use of the structure-directing agent in an amount within this range, the titanosilicate (I) can be prepared easily.

The contact of the titanosilicate (II) with the structure-directing agent may be performed by the following method: the titanosilicate (II) and the structure-directing agent are placed in a tightly closed container such as an autoclave and pressurized with heating; or the titanosilicate (II) and the structure-directing agent are mixed with or without stirring in a container such as a glass flask in atmosphere.

The contact is performed at a temperature of preferably 0° C., more preferably 20° C., even more preferably 50° C., particularly preferably 100° C., as the lower limit and at a temperature of approximately 250° C., preferably 200° C., more preferably 180° C., as the upper limit.

The contact is performed at any pressure without particular limitations and usually performed at approximately 0 to 10 MPa in terms of gage pressure. The titanosilicate (I) obtained by the contact is usually separated by filtration. The separated titanosilicate (I) may be subjected, if necessary, to post-treatment such as washing and drying. Presumably, this post-treatment can also adjust the amount of the structure-directing agent in the obtained titanosilicate (I).

In the present invention, the titanosilicate (I) is preferably obtained by further washing after the contact. This washing presumably not only enhances the purity of the obtained titanosilicate (I) but also adjusts the amount of the structure-directing agent present in the titanosilicate (I). The washing may be performed by appropriately adjusting the amount, pH, etc., of the wash, if necessary. The washing is preferably performed with water as a wash, more preferably until the pH of the wash is 7 to 11. When drying is performed after the contact, its conditions including a temperature can be set appropriately within a range that does not impair the characteristics of the titanosilicate (I) shown below.

In this context, the titanosilicate (I) is converted to an MWW structure by calcination and therefore classified into a Ti-MWW precursor.

Examples of the titanosilicate (II) include crystalline titanosilicate having an MWW or MSE structure, a Ti-MWW precursor (a), and Ti-YNU-1.

Examples of the Ti-YNU-1 include Ti-YNU-1 described in Angewandte Chemie International Edition 43, 236-240, (2004).

Examples of the crystalline titanosilicate having an MWW structure include Ti-MWW described in Japanese Patent Laid-Open No. 2003-327425. Examples of the crystalline titanosilicate having an MSE structure include Ti-MCM-68 described in Japanese Patent Laid-Open No. 2008-50186.

In the present specification, the Ti-MWW precursor means titanosilicate having a laminar structure. The Ti-MWW precursor exhibits Ti-MWW properties by calcination. The calcination will be described later.

Any titanosilicate in a laminar form that is converted to Ti-MWW by calcination can be used as the Ti-MWW precursor (a) without particular limitations. The Ti-MWW precursor (a) preferably has a molar ratio of silicon to nitrogen (Si/N ratio) of 21 or larger. The titanosilicate (I) can also be used as the Ti-MWW precursor (a).

Examples of the Ti-MWW precursor (a) include Ti-MWW precursors described in Japanese Patent Laid-Open No. 2005-262164.

The titanosilicate (II) is preferably crystalline titanosilicate having an MWW or MSE structure, or a Ti-MWW precursor (a), more preferably Ti-MWW having an MWW structure, or a Ti-MWW precursor (a).

The titanosilicate (II) can be produced by a method known in the art such as methods described in the documents. The crystalline titanosilicate having an MWW structure can also be produced, for example, by calcining the Ti-MWW precursor (a).

Examples of typical methods for producing the Ti-MWW precursor (a) include the following first to third aspects.

The first aspect is a production method comprising the following steps 1 and 2.

### Step 1

In the step 1, a mixture containing a structure-directing agent, a compound containing an element belonging to group 13 in the periodic table of the elements (hereinafter, this compound is referred to as a “13 group element-containing compound”), a silicon-containing compound, a titanium-containing compound, and water is heated to obtain a laminar compound.

### Step 2

In the step 2, the laminar compound obtained in the step 1 is acid-treated to obtain a Ti-MWW precursor (a).

In this context, the laminar compound is called an as-synthesized sample. This sample is directly converted by calcination to zeolite having an MWW structure. However, for the laminar compound, an UV-visible absorption spectrum of a wavelength region of 200 nm to 500 nm does not have the greatest absorption peak in a wavelength region of 220±10 nm. Therefore, the laminar compound is not titanosilicate and is definitively distinguished from the Ti-MWW precursor (see e.g., Chemistry Letters 774-775 (2000)).

Examples of the structure-directing agent in the step 1 include the same compounds as those used for the preparation of the titanosilicate (I). These compounds may be used alone or as a mixture of two or more thereof at an arbitrary ratio.

The structure-directing agent is preferably piperidine or hexamethyleneimine.

In the mixture in the step 1, the structure-directing agent is used in an amount ranging from preferably 0.1 to 5 mol, more preferably 0.5 to 3 mol, with respect to 1 mol of silicon in the silicon-containing compound.

Examples of the 13 group element-containing compound include boron-containing, aluminum-containing, and gallium-containing compounds. The boron-containing compound is preferable.

Examples of the boron-containing compound include: boric acid; borate; boron oxide; boron halide; and trialkylboron compounds which have an alkyl group having 1 to 4 carbon atoms. Particularly, boric acid is preferable.

Examples of the aluminum-containing compound include sodium aluminate. Examples of the gallium-containing compound include gallium oxide.

In the mixture in the step 1, the 13 group element-containing compound is used in an amount ranging from preferably 0.01 to 10 mol, more preferably 0.1 to 5 mol, with respect to 1 mol of silicon in the silicon-containing compound.

Examples of the silicon-containing compound include silicic acid, silicate, silicon oxide, silicon halide, fumed silica compounds, tetraalkyl orthosilicate, and colloidal silica. The fumed silica compounds are preferable.

In the mixture in the step 1, water is used at a proportion ranging from preferably 5 to 200 mol, more preferably 10 to 50 mol, with respect to 1 mol of silicon in the silicon-containing compound.

Examples of the titanium-containing compound include titanium alkoxide, titanate, titanium oxide, titanium halide, inorganic acid salts of titanium, and organic acid salts of titanium. The titanium alkoxide is preferable.

Example of the titanium alkoxide include compounds which have an alkoxyl group having 1 to 4 carbon atoms, for example, titanium tetramethoxide, titanium tetraethoxide, titanium tetraisopropoxide, and titanium tetrabutoxide.

Examples of the organic acid salts of titanium include titanium acetate. Examples of the inorganic acid salts of titanium include titanium nitrate, titanium sulfate, titanium phosphate, and titanium perchlorate. Examples of the titanium halide include titanium tetrachloride. Examples of the titanium oxide include titanium dioxide.

In the mixture in the step 1, the titanium-containing compound is usually used in an amount ranging from 0.005 to 0.1 mol, more preferably 0.01 to 0.05 mol, with respect to 1 mol of silicon in the silicon-containing compound.

The heating procedure in the step 1 is preferably performed as follows: the mixture is placed in a tightly closed container such as an autoclave and subjected to hydrothermal synthesis conditions involving pressurization with heating (see e.g., Chemistry Letters 774-775 (2000)). The heating procedure is performed at a temperature ranging from preferably 110° C. to 200° C., more preferably 120° C. to 180° C. The mixture thus heated is usually separated into solid and liquid components by filtration. The redundant raw materials in the mixture thus heated are filtered off. Furthermore, the solid component is washed with water or the like and dried by heating to obtain the laminar compound. In this context, the solid component is preferably washed until the pH of the wash is 7 to 11. The drying by heating is preferably performed at a temperature of approximately 0° C. to 100° C. until no decrease in the weight of the solid component is seen.

Next, the step 2 will be described.

In the step 2, the laminar compound obtained in the step 1 is acid-treated to obtain a Ti-MWW precursor (a).

The “acid treatment” herein means contact with an acid and specifically means contact of the compound to be treated with a solution containing an acid or with an acid itself. The contact can be performed by any method without limitations and may be performed by the following method: the acid or the acid solution is sprayed or applied to the compound to be treated; or the compound to be treated is immersed in the acid or the acid solution. The method is preferable, wherein the compound to be treated is immersed in the acid or the acid solution.

The acid used in the acid treatment may be an inorganic or organic acid. Examples of the inorganic acid include nitric acid, hydrochloric acid, sulfuric acid, perchloric acid, and fluorosulfonic acid. Examples of the organic acid include formic acid, acetic acid, propionic acid, and tartaric acid. In the acid treatment, these acids may be used alone or in combination of two or more thereof.

The acid solution can be prepared, for example, by dissolving the organic or inorganic acid salt in a solvent. Examples of the solvent include water, alcohol, ether, ester, ketone, and mixtures thereof. Particularly, water is preferable.

The acid is used at any concentration without particular limitations and is usually used in a range of 0.01 M to 20 M (M: mol/l). The concentration of the inorganic acid is preferably 1 M to 5 M.

The contact of the laminar compound with the acid is performed at any temperature without limitations and usually performed at 0° C. to 200° C., preferably 50° C. to 180° C., more preferably 60° C. to 150° C.

The second aspect for producing the Ti-MWW precursor (a) is a method comprising the following steps I to IV.

### Step I

In the step I, a mixture containing a structure-directing agent, a 13 group element-containing compound, a silicon-containing compound, and water is heated to obtain a solid product a.

### Step II

In the step II, the solid product a is acid-treated to obtain a solid product b.

### Step III

In the step III, a structure-directing agent, a titanium-containing compound, and water are added to the solid product b, and the obtained mixture is heated to obtain a solid product c.

### Step IV

In the step IV, the solid product c is acid-treated to obtain a Ti-MWW precursor (a).

Examples of the structure-directing agent in the step I include the same compounds as those used for the preparation of the titanosilicate (I). The structure-directing agent is preferably piperidine or hexamethyleneimine. These compounds may be used alone or as a mixture of two or more thereof at an arbitrary ratio.

In the mixture in the step I, the structure-directing agent is used in an amount ranging from preferably 0.1 to 5 mol, more preferably 0.5 to 3 mol, with respect to 1 mol of silicon in the silicon-containing compound.

Examples of the 13 group element-containing compound and the silicon-containing compound in the step I respectively include the same compounds as those used for the preparation in the first aspect.

In the mixture in the step I, the 13 group element-containing compound is used in an amount ranging from preferably 0.01 to 10 mol, more preferably 0.1 to 5 mol, with respect to 1 mol of silicon contained in the silicon-containing compound.

In the mixture in the step I, water is used at a proportion ranging from preferably 5 to 200 mol, more preferably 10 to 50 mol, with respect to 1 mol of silicon in the silicon-containing compound.

The heating procedure in the step I can be performed in the same manner as that in the step 1 in the first aspect.

Alternatively, a step I-2 shown below can also be performed between the steps I and II. In this case, a solid product a1 obtained in the step I-2 is used in the step II instead of the solid product a obtained in the step I.

### Step I-2

In the step I-2, the solid product a is calcined.

Calcination is one mode of high-temperature treatment of minerals aimed at chemical reaction, sintering, or thermal decomposition such as dehydrative condensation, etc. (see Chemical Dictionary, KYORITSU SHUPPAN CO., LTD, 1960) and is generally distinguished from drying aimed at moisture removal. In the present invention, the calcination is aimed at dehydration condensation between the layers of the laminar compound. The calcination is performed in nonliquid phase so that it can be distinguished from the heat treatment performed in liquid phase. The calcination for the preparation of the Ti-MWW precursor (a) may not result in complete dehydrative condensation.

The calcination can be performed under conditions known in the art and may be performed in an open system or gas flow system. The calcination is performed most easily in the presence of air. Alternatively, the calcination may be performed by introducing oxygen thereto after heating to a predetermined temperature in an inert gas (e.g., nitrogen) atmosphere.

In the present specification, the calcination temperature ranges from preferably higher than 200° C. to 1000° C. or lower, more preferably 300° C. to 650° C. The calcination performed at too low a temperature may require very long time for achieving the aim. By contrast, the calcination performed at too high a temperature may cause structural destruction.

Next, the step II will be described. In the step II, the solid product a or a1 is acid-treated to obtain a solid product b. The acid treatment in the step II can be performed in the same manner as that in the first aspect.

Alternatively, a step II-2 shown below can also be performed between the steps II and III. In this case, a solid product b1 obtained in the step II-2 is used in the step III instead of the solid product b.

### Step II-2

Step of calcining the solid product b.

The present step can be performed under the same conditions as those in the step I-2.

Next, the step III will be described. In the step III, a structure-directing agent, a titanium-containing compound, and water are added to the solid product b or b1, and the obtained mixture is heated to obtain a solid product c.

Examples of the structure-directing agent and the titanium-containing compound in the step III respectively include the same compounds as those used for the first aspect. These compounds may be used alone or as a mixture of two or more thereof at an arbitrary ratio.

In the mixture in the step III, the structure-directing agent is used in an amount ranging from preferably 0.1 to 5 mol, more preferably 0.5 to 3 mol, with respect to 1 mol of silicon in the solid product b or b1.

In the mixture in the step III, the titanium-containing compound is usually used in an amount ranging from 0.005 to 0.1 mol, more preferably 0.01 to 0.05 mol, with respect to 1 mol of silicon in the solid product b or b1.

In the mixture in the step III, water added to the solid product b or b1 is used at a proportion ranging from preferably 5 to 200 mol, more preferably 10 to 50 mol, with respect to 1 mol of silicon in the solid product b.

The heating procedure in the step III can be performed in the same manner as that in the first aspect.

Next, the step IV will be described. In the step IV, the solid product c is acid-treated to obtain a Ti-MWW precursor (a).

The acid treatment in the step IV can be performed in the same manner as that in the first aspect.

The third aspect for producing the Ti-MWW precursor (a) is a method comprising the following steps A and B.

### Step A

In the step A, a mixture containing a structure-directing agent, a 13 group element-containing compound, a silicon-containing compound, a titanium-containing compound, and water is heated to obtain a laminar compound i.

### Step B

In the step B, the laminar compound i is contacted with a titanium-containing compound and an inorganic acid to obtain a Ti-MWW precursor (a).

Examples of the structure-directing agent, the 13 group element-containing compound, the silicon-containing compound, and the titanium-containing compound in the step A respectively include the same compounds as those used for the first aspect.

In the mixture in the step A, the structure-directing agent, the 13 group element-containing compound, the silicon-containing compound, and the titanium-containing compound are used in the same amounts as those in the step 1 in the first aspect.

The heating procedure in the step A can be performed in the same manner as that in the step 1.

A step A-2 shown below can also be performed instead of the step A. In this case, a solid product a obtained in the step A-2 is used in the step B instead of the laminar compound i.

### Step A-2

In the step A-2, a mixture containing the structure-directing agent, the 13 group element-containing compound, the silicon-containing compound, and water is heated to obtain a solid product a.

The step A-2 can be performed in the same manner as the step I in the second aspect.

Next, the step B will be described. In the step B, the laminar compound i or the solid product a is contacted with a titanium-containing compound and an inorganic acid to obtain a Ti-MWW precursor (a).

Examples of the inorganic acid in the step B include sulfuric acid, hydrochloric acid, nitric acid, perchloric acid, fluorosulfonic acid, and mixtures thereof. The nitric acid, perchloric acid, fluorosulfonic acid, and mixtures thereof are preferable. When the acid is used in a solution, examples of a solvent thereof include water, alcohol, ether, ester, and ketone. Particularly, water is preferable. The inorganic acid is used at any concentration without particular limitations and generally used in a range of 0.01 M to 20 M (M: mol/l). The concentration of the inorganic acid is preferably 1 M to 5 M.

Examples of the titanium-containing compound in the step B include the same compounds as those used for the step I. The titanium-containing compound is usually used in an amount ranging from 0.001 to 10 parts by weight, preferably 0.01 to 2 parts by weight, with respect to 1 part by weight of the laminar compound i or the solid product a.

The contact of the laminar compound i or the solid product a with the titanium-containing compound and the inorganic acid is usually performed by contacting the laminar compound i or the solid product a with a mixture of the titanium-containing compound and the inorganic acid at a temperature of preferably 20° C. to 150° C., more preferably 50° C. to 104° C. The contact is performed at any pressure without limitations and usually performed at approximately 0 to 10 MPa in terms of gage pressure.

The titanosilicate (I) and the silylated form thereof can respectively be used as a catalyst for oxidation reaction of an organic compound. One aspect of the present invention encompasses a catalyst for oxidation reaction of an organic compound, comprising the titanosilicate (I) or the silylated form thereof. The catalyst of the present invention is useful in oxidation reaction of an organic compound, particularly, epoxidation reaction of olefin.

The titanosilicate of the present invention and the silylated form thereof can respectively be used as a catalyst, in the same manner as the titanosilicate (I), in the method for producing an oxidized compound.

In the production method of the present invention, an organic compound is reacted with an oxidizing agent in the presence of the titanosilicate (I) or the silylated form thereof.

In the present invention, the oxidizing agent means a compound that imparts oxygen atoms to the organic compound.

Examples of the oxidizing agent include oxygen and peroxide. Examples of the peroxide include hydrogen peroxide and organic peroxide.

Examples of the organic peroxide include t-butyl hydroperoxide, di-t-butyl peroxide, t-amyl hydroperoxide, cumene hydroperoxide, methylcyclohexyl hydroperoxide, tetralin hydroperoxide, isobutylbenzene hydroperoxide, ethylnaphthalene hydroperoxide, and peracetic acid. These peroxides can also be used as a mixture of two or more thereof.

In particular, the peroxide is preferably hydrogen peroxide. In the production method, the hydrogen peroxide is used in a form of an aqueous solution containing hydrogen peroxide at a concentration ranging from 0.0001% by weight or higher to lower than 100% by weight. The hydrogen peroxide can be produced by a method known in the art or may be a commercially available product or a product produced from oxygen and hydrogen in the presence of a noble metal in the same reaction system as that of the oxidation reaction.

In the present invention, the oxidizing agent can be used in an amount arbitrarily selected according to the kind of the organic compound, reaction conditions, etc., and is used in amount of preferably 0.01 parts by weight or larger, more preferably 0.1 parts by weight or larger, with respect to 100 parts by weight of the organic compound. The amount of the oxidizing agent is preferably 1000 parts by weight, more preferably 100 parts by weight, as the upper limit with respect to 100 parts by weight of the organic compound.

Examples of the organic compound in the production method include an aromatic compound such as benzene and a phenol compound, and an olefin compound.

Examples of the phenol compound include unsubstituted or substituted phenol. In this context, the substituted phenol means alkylphenol which has, as a substituent, a linear or branched alkyl group having 1 to 6 carbon atoms, or a cycloalkyl group. Examples of the linear or branched alkyl group include methyl, ethyl, isopropyl, butyl, and hexyl groups. Examples of the cycloalkyl group include a cyclohexyl group.

Specific examples of the phenol compound include 2-methylphenol, 3-methylphenol, 2,6-dimethylphenol, 2,3,5-trimethylphenol, 2-ethylphenol, 3-isopropylphenol, 2-butylphenol, and 2-cyclohexylphenol. Particularly, phenol is preferable.

Examples of the olefin compound include compounds having a substituted or unsubstituted hydrocarbyl group or hydrogen bonded to carbon atoms constituting the olefin double bond.

Examples of the substituent for the hydrocarbyl group include hydroxy groups, halogen atoms, carbonyl groups, alkoxycarbonyl groups, cyano groups, and nitro groups. Examples of the hydrocarbyl group include saturated hydrocarbyl groups. Examples of the saturated hydrocarbyl group include alkyl groups.

Specific examples of the olefin compound include alkene having 2 to 10 carbon atoms and cycloalkene having 4 to 10 carbon atoms.

Examples of the alkene having 2 to 10 carbon atoms include ethylene, propylene, butene, pentene, hexene, heptene, octene, nonene, decene, 2-butene, isobutene, 2-pentene, 3-pentene, 2-hexene, 3-hexene, 4-methyl-1-pentene, 2-heptene, 3-heptene, 2-octene, 3-octene, 2-nonene, 3-nonene, 2-decene, and 3-decene.

Examples of the cycloalkene having 4 to 10 carbon atoms include cyclobutene, cyclopentene, cyclohexene, cycloheptene, cyclooctene, cyclononene, and cyclodecane.

In the present invention, the organic compound is preferably an olefin compound, more preferably alkene having 2 to 10 carbon atoms, even more preferably alkene having 2 to 5 carbon atoms, particularly preferably propylene.

In the present invention, the organic compound can be used in an amount arbitrarily selected according to its kind, reaction conditions, etc., and is used in an amount of preferably 0.01 part by weight or larger, more preferably 0.1 part by weight or larger, with respect to 100 parts by weight of the total amount of the solvent in the liquid phase. The amount of the organic compound is preferably 1000 parts by weight, more preferably 100 parts by weight, as the upper limit with respect to 100 parts by weight of the total amount of the solvent in the liquid phase.

In the production method of the present invention, the titanosilicate (I) or the silylated form thereof can be used in an amount appropriately selected according to the type of the reaction and is generally used in an amount of 0.01% by weight, preferably 0.1% by weight, more preferably 0.5% by weight, as the lower limit with respect to the total amount of the solvent in the liquid phase and in an amount of 20% by weight, preferably 10% by weight, more preferably 8% by weight, as the upper limit with respect to the total amount of the solvent in the liquid phase.

Examples of the oxidation reaction in the present invention include epoxidation reaction of the olefin compound and hydroxylation reaction of the aromatic compound such as benzene or a phenol compound.

Examples of the epoxidation reaction include reaction through which the olefin compound is converted to a corresponding epoxy compound.

Examples of the hydroxylation reaction include reaction through which the aromatic compound is converted by the hydroxylation of its aromatic ring to a phenol or polyhydric phenol compound.

The production method of the present invention is suitable for reaction through which alkene having 2 to 10 carbon atoms, preferably alkene having 2 to 5 carbon atoms, particularly propylene is epoxidized using hydrogen peroxide as the oxidizing agent.

In the production method of the present invention, the oxidized compound means an oxygen-containing compound obtained through the oxidation reaction. Examples of the oxidized compound include epoxy compounds obtained through the epoxidation reaction and phenol or polyhydric phenol compounds obtained through the hydroxylation reaction.

In the production method of the present invention, the titanosilicate (I) can also be contacted with hydrogen peroxide in advance and then subjected to the reaction.

The hydrogen peroxide in the contact can be used in a form of a hydrogen peroxide solution. The hydrogen peroxide solution usually has a hydrogen peroxide concentration ranging from 0.0001% by weight to 50% by weight. The hydrogen peroxide solution may be an aqueous solution or a solution obtained using a solvent other than water. The solvent other than water can be selected as suitable one from among, for example, solvents for the oxidation reaction. The contact is usually performed at a temperature ranging from 0° C. to 100° C., preferably 0° C. to 60° C.

In the production method of the present invention, when the oxidizing agent is hydrogen peroxide, the hydrogen peroxide produced in the same reaction system as that of the oxidation reaction may be supplied for the reaction.

The hydrogen peroxide, when produced in the same reaction system as that of the oxidation reaction, can be produced, for example, from oxygen and hydrogen in the presence of a noble metal catalyst.

Examples of the noble metal catalyst include noble metals such as palladium, platinum, ruthenium, rhodium, iridium, osmium, and gold, and alloys or mixtures thereof. Preferable examples of the noble metal include palladium, platinum, and gold. The noble metal is more preferably palladium. For example, colloidal palladium may be used as the palladium (see e.g., Example 1 in Japanese Patent Laid-Open No. 2002-294301). The noble metal catalyst used may be a noble metal compound that is converted to a noble metal by reduction in the oxidation reaction system. The noble metal catalyst is preferably palladium compound.

When palladium is used as the noble metal catalyst, an additional metal other than palladium, such as platinum, gold, rhodium, iridium, and osmium can also be added thereto and used as a mixture. Preferable examples of the metal other than palladium include platinum.

Examples of the palladium compound include tetravalent and divalent palladium compounds.

Examples of tetravalent palladium compound include sodium hexachloropalladate (IV) and potassium hexachloropalladate (IV). Examples of divalent palladium compound include palladium (II) chloride, palladium (II) bromide, palladium (II) acetate, palladium (II) acetylacetonate, dichlorobis(benzonitrile)palladium (II), dichlorobis(acetonitrile)palladium (II), dichloro(bis(diphenylphosphino)ethane)palladium (II), dichlorobis(triphenylphosphine)palladium (II), tetraamminepalladium (II) chloride, tetraamminepalladium (II) bromide, dichloro(cycloocta-1,5-diene)palladium (II), and palladium (II) trifluoroacetate.

The noble metal is usually supported by a carrier for use. The noble metal can be supported, for use, by the titanosilicate (I) or by oxide (e.g., silica, alumina, titania, zirconia, and niobia), hydrate (e.g., niobic acid, zirconic acid, tungstic acid, and titanic acid), carbon, and mixtures thereof. When the noble metal is supported by the carrier other than the titanosilicate (I), the carrier comprising the noble metal supported thereby is mixed with the titanosilicate (I), and this mixture can be used as a catalyst. Among the carriers other than the titanosilicate (I), preferable examples thereof include carbon. Known carbon carriers are active carbon, carbon black, graphite, carbon nanotube, etc.

The noble metal-supported catalyst is prepared by a known method, for example, by supporting a noble metal compound onto a carrier, followed by reduction. The noble metal compound can be supported by a method conventionally known in the art such as impregnation.

The reduction method may be reduction using a reducing agent such as hydrogen or reduction using ammonia gas generated during thermal decomposition in an inert gas atmosphere. The reduction temperature differs depending on the kind, etc., of the noble metal compound and is usually 100° C. to 500° C., preferably 200° C. to 350° C., for tetraamminepalladium (II) chloride used as the noble metal compound.

The noble metal-supported catalyst usually comprises the noble metal in a range of 0.01 to 20% by weight, preferably 0.1 to 5% by weight.

The amount of the noble metal is generally 0.001 weight parts, preferably 0.01 weight parts, more preferably 0.1 weight parts with respect to 100 parts of the titanosilicate (I), as the lower limit of that. The amount of the noble metal is generally 100 weight parts, preferably 20 weight parts, more preferably 5 weight parts with respect to 100 parts of the titanosilicate (I), as the upper limit of that.

In the present invention, conditions including a reaction temperature and a reaction pressure can be set arbitrarily according to the kinds, amounts, etc., of the materials used.

The reaction temperature is preferably 0° C., more preferably 40° C., as the lower limit and is preferably 200° C., more preferably 150° C., as the upper limit.

The reaction pressure is preferably 0.1 MPa, more preferably 1 MPa, as the lower limit and is preferably 20 MPa, more preferably 10 MPa, as the upper limit.

The reaction product can be collected by a method known in the art such as separation by distillation.

Hereinafter, the production method of the present invention will be described in detail by taking, as an example, a method for producing an epoxy compound by oxidation (epoxidation) of an olefin compound.

In this production method, the reaction is usually performed in a liquid phase containing a solvent. Examples of the solvent include water, an organic solvent, and mixtures of water and the organic solvent.

Examples of the organic solvent include alcohol, ketone, nitrile, ether, aliphatic hydrocarbon, aromatic hydrocarbon, halogenated hydrocarbon, ester and mixtures thereof.

Examples of the aliphatic hydrocarbon include aliphatic hydrocarbons having 5 to 10 carbon atoms, such as hexane and heptane. Examples of the aromatic hydrocarbon include aromatic hydrocarbons having 6 to 15 carbon atoms, such as benzene, toluene, and xylene.

Examples of the alcohol include monohydric alcohol having 1 to 6 carbon atoms and glycol having 2 to 8 carbon atoms. The alcohol is preferably aliphatic alcohol having 1 to 8 carbon atoms, more preferably monohydric alcohol having 1 to 4 carbon atoms, such as methanol, ethanol, isopropanol, and t-butanol, even more preferably t-butanol.

The nitrile is preferably C₂ to C₄ alkylnitrile (e.g., acetonitrile, propionitrile, isobutyronitrile, and butyronitrile) and benzonitrile, most preferably acetonitrile.

The organic solvent is preferably alcohol or nitrile from the viewpoint of catalyst activities and selectivity.

In the method for producing an epoxy compound, the presence of a buffer in the reaction system can prevent decrease in catalyst activities, further enhance catalyst activities, or improve the use efficiency of a source gas.

The buffer is generally present in the reaction system in the manner that it is dissolved in the liquid phase. When hydrogen peroxide produced in the same reaction system as the epoxidation is used as the oxidizing agent, the buffer may be contained in a portion of the noble metal complex in advance. In one method, for example, an ammine complex such as tetraamminepalladium (II) chloride is supported onto a carrier by impregnation and then reduced to form residual ammonium ions such that the buffer is generated during the epoxidation reaction. The buffer is usually added in an amount of 0.001 mmol/kg to 100 mmol/kg per kg of the solvent in the liquid phase.

Examples of the buffer include buffers comprising: 1) an anion selected from the group consisting of sulfuric acid ions, hydrogen sulfate ions, carbonic acid ions, hydrogen carbonate ions, phosphoric acid ions, hydrogen phosphate ions, dihydrogen phosphate ions, hydrogen pyrophosphate ions, pyrophosphoric acid ions, halogen ions, nitric acid ions, hydroxide ions, and C₁ to C₁₀ carboxylic acid ions and 2) a cation selected from the group consisting of ammonium, C₁ to C₂₀ alkylammonium, C₇ to C₂₀ alkylarylammonium, alkali metals, and alkaline-earth metals.

Examples of the C₁ to C₁₀ carboxylic acid ions include acetic acid, formic acid, propionic acid, butyric acid, valeric acid, caproic acid, caprylic acid, capric acid, and benzoic acid ions.

Examples of the alkylammonium ions include tetramethylammonium, tetraethylammonium, tetra-n-propylammonium, tetra-n-butylammonium, and cetyltrimethylammonium. Examples of the alkali metal and alkaline-earth metal cations include lithium, sodium, potassium, rubidium, cesium, magnesium, calcium, strontium, and barium cations.

Preferable examples of the buffer include: ammonium salts of inorganic acids, such as ammonium sulfate, ammonium hydrogen sulfate, ammonium carbonate, ammonium hydrogen carbonate, diammonium hydrogen phosphate, ammonium dihydrogen phosphate, ammonium phosphate, ammonium hydrogen pyrophosphate, ammonium pyrophosphate, ammonium chloride, and ammonium nitrate; and ammonium salts of C₁ to C₁₀ carboxylic acids, such as ammonium acetate. Preferable examples of the ammonium salts include ammonium dihydrogen phosphate.

In the method for producing an epoxy compound, when hydrogen peroxide for use is synthesized from oxygen and hydrogen in the same reaction system as that of the oxidation reaction, the presence of a quinoid compound in the reaction system can further enhance oxidized compound selectivity.

Examples of the quinoid compound include a ρ-quinoid compound represented by the following formula (1) and a phenanthraquinone compound:

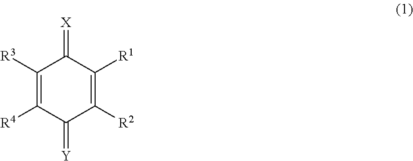

wherein R¹, R², R³, and R⁴ represent a hydrogen atom, or R¹ and R² are bonded to each other at their ends and represent, together with the carbon atoms bonded thereto, a naphthalene ring which may be substituted, or R³ and R⁴ are bonded to each other at their ends and represent, together with the carbon atoms bonded thereto, a naphthalene ring which may be substituted; and X and Y are the same as or different from each other and represent an oxygen atom or an NH group.

Examples of the compound of the formula (1) include

1) a quinone compound (1A) represented by the formula (1) wherein R¹, R², R³, and R⁴ are a hydrogen atom, and both X and Y are an oxygen atom;
2) a quinoneimine compound (1B) represented by the formula (1) wherein R¹, R², R³, and R⁴ are a hydrogen atom, and X and Y are an oxygen atom and an NH group, respectively; and
3) a quinonediimine compound (1C) represented by the formula (1) wherein R¹, R², R³, and R⁴ are a hydrogen atom, and X and Y are an NH group.

The quinoid compound of the formula (1) encompasses the following anthraquinone compound (2):

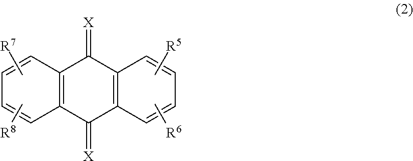

wherein X and Y are as defined in the formula (1); and R⁵, R⁶, R⁷, and R⁸ are the same as or different from each other and represent a hydrogen atom, a hydroxyl group, or an alkyl group (e.g., C₁ to C₆ alkyl groups such as methyl, ethyl, propyl, butyl, and pentyl).

In the formulas (1) and (2), X and Y preferably represent an oxygen atom.

The dihydro forms of quinoid compounds which have been partially hydrogenated may be formed in a certain reaction condition. Such dihydro forms can be used for the epoxidation.

Examples of the quinoid compound include benzoquinone, naphthoquinone, anthraquinone, alkylanthraquinone compounds, polyhydroxyanthraquinone, ρ-quinoid compounds, and o-quinoid compounds.

Examples of the alkylanthraquinone compounds include 2-alkylanthraquinone compounds such as 2-ethylanthraquinone, 2-t-butylanthraquinone, 2-amylanthraquinone, 2-methylanthraquinone, 2-butylanthraquinone, 2-t-amylanthraquinone, 2-isopropylanthraquinone, 2-s-butylanthraquinone, and 2-s-amylanthraquinone; and polyalkylanthraquinone compounds such as 1,3-diethylanthraquinone, 2,3-dimethylanthraquinone, 1,4-dimethylanthraquinone, and 2,7-dimethylanthraquinone. Examples of the polyhydroxyanthraquinone include 2,6-dihydroxyanthraquinone. Examples of the ρ-quinoid compounds include naphthoquinone and 1,4-phenanthraquinone. Examples of the o-quinoid compounds include 1,2-, 3,4-, and 9,10-phenanthraquinones.

Preferable examples of the quinoid compound include: anthraquinone; and 2-alkylanthraquinone compounds represented by the formula (2) wherein X and Y are an oxygen atom, R⁵ is an alkyl group substituted at position 2, and R⁶, R⁷, and R⁸ represent a hydrogen atom.

The quinoid compound can usually be used in an amount ranging from 0.001 mmol/kg to 500 mmol/kg per kg of the solvent in the liquid phase.

The amount of the quinoid compound is preferably 0.01 mmol/kg to 50 mmol/kg.

In the method of the present invention, a salt of ammonium, alkylammonium, or alkylarylammonium can also be added simultaneously with the quinoid compound to the reaction system.

The quinoid compound can also be prepared by oxidizing a dihydro form of the quinoid compound using oxygen or the like in the reaction system. For example, a hydrogenated quinoid compound such as hydroquinone or 9,10-anthracenediol is added to the liquid phase and oxidized with oxygen in the reaction system to form the quinoid compound, which may then be used.

Examples of the dihydro form of the quinoid compound include compounds represented by the following formulas (3) and (4), which are dihydro forms of the compounds of the formulas (1) and (2):

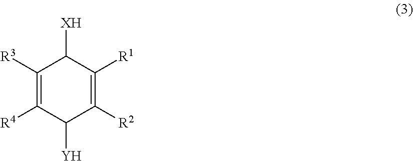

wherein R¹, R², R³, R⁴, X, and Y are as defined in the formula (1), and

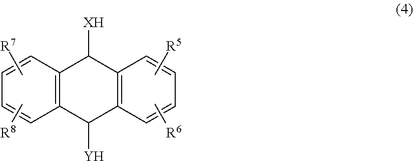

wherein X, Y, R⁵, R⁶, R⁷, and R⁸ are as defined in the formula (2).

In the formulas (3) and (4), X and Y preferably represent an oxygen atom.

Preferable examples of the dihydro form of the quinoid compound include dihydro forms corresponding to the preferable quinoid compounds.

Examples of the reaction method in the method for producing an epoxy compound include fixed-bed flow reaction and perfect mixing flow reaction of slurry.

The olefin compound can be oxidized for epoxidation in any reaction gas atmosphere without limitations using the peroxide produced in advance.

When the peroxide is produced from oxygen and hydrogen in the presence of the noble metal in the same reaction system as that of the oxidation reaction, oxygen and hydrogen are usually supplied to a reactor at a partial pressure ratio ranging from 1:50 to 50:1. The partial pressure ratio between oxygen and hydrogen is preferably oxygen:hydrogen=1:2 to 10:1. At too high a partial pressure ratio between oxygen and hydrogen (oxygen/hydrogen), the rate of epoxy compound production may be decreased. By contrast, at too low a partial pressure ratio between oxygen and hydrogen (oxygen/hydrogen), epoxy compound selectivity may be decreased due to increased by-products of alkane compounds.

In the present reaction, the oxygen and hydrogen gases may be diluted. Examples of a gas used in the dilution include nitrogen, argon, carbon dioxide, methane, ethane, and propane. The gas for the dilution is used at any concentration without limitations.

Examples of the oxygen as a raw material include oxygen gas and air. The oxygen gas used can be oxygen gas produced by an inexpensive pressure swing method or, if necessary, highly pure oxygen gas produced by cryogenic separation or the like.

The present epoxidation is usually performed at a reaction temperature of 0° C., preferably 40° C., more preferably 50° C., as the lower limit and at a reaction temperature of 200° C., preferably 150° C., more preferably 120° C., as the upper limit.

At too low a reaction temperature, the reaction rate is slowed down. By contrast, at too high a reaction temperature, by-products are increased due to side reaction.

The reaction is performed at any pressure without particular limitations and usually performed at 0.1 MPa to 20 MPa, preferably 1 MPa to 10 MPa, in terms of gage pressure. The reaction product can be collected by a method known in the art such as separation by distillation.

In the present epoxidation, the titanosilicate (I) or the silylated form thereof can be used in an amount appropriately selected according to the type of the reaction and is usually used in an amount of 0.01% by weight, preferably 0.1% by weight, more preferably 0.5% by weight, as the lower limit with respect to the total amount of the solvent in the liquid phase and in an amount of 20% by weight, preferably 10% by weight, more preferably 8% by weight, as the upper limit with respect to the total amount of the solvent in the liquid phase.

In the present epoxidation, the olefin compound can be used in an amount appropriately selected according to its kind, reaction conditions, etc., and is usually used in an amount of 0.01 parts by weight, preferably 0.1 parts by weight, more preferably 1 part by weight, as the lower limit with respect to 100 parts by weight of total amount of the solvent in the liquid phase and in an amount of 1000 parts by weight, preferably 100 parts by weight, more preferably 50 parts by weight, as the upper limit with respect to 100 parts by weight of total amount of the solvent in the liquid phase.

In the present epoxidation, the oxidizing agent can be used in an amount arbitrarily selected according to the kind of the olefin compound, reaction conditions, etc., and is used in an amount of preferably 0.1 parts by weight or larger, more preferably 1 part by weight or larger, with respect to 100 parts by weight of the olefin compound. The amount of the oxidizing agent is preferably 100 parts by weight, more preferably 50 parts by weight, as the upper limit with respect to 100 parts by weight of the olefin compound.

Hereinafter, the present invention will be described with reference to Examples.

In Examples of the present specification, each measurement was performed according to the following method.

### 1. Ti (Titanium), Si (Silicon), and B (Boron) Contents

These contents were measured by alkali fusion, dissolution in nitric acid, and ICP emission spectroscopy using SUMIGRAPH NCH-22F model (manufactured by Sumika Chemical Analysis Service, Ltd.).

### 2. N (Nitrogen) Content

The N content was measured by oxygen circulating combustion and TCD detection systems using SUMIGRAPH NCH-22F model (manufactured by Sumika Chemical Analysis Service, Ltd.).

### 3. UV-Visible Absorption Spectrum (UV-Vis Spectrum)

The UV-Vis spectrum was measured by a diffuse reflection method using an UV-visible spectrophotometer (manufactured by JASCO Corp. (V-7100)) equipped with a diffuse reflection accessory (Praying Mantis manufactured by HARRICK Scientific Products).

Measurement range: 200 to 500 nm
Standard for baseline: Spectralon

### 4. Specific Surface Area (SN₂) by Nitrogen Adsorption

Approximately 100 mg of a sample was degassed at 150° C. for 8 hours. A nitrogen adsorption isotherm was then measured at constant volume at an adsorption temperature of 77 K using BELSORP-mini (manufactured by BEL JAPAN INC.), and the specific surface area was calculated by a multi-point BET method.

In this multi-point BET method, at least three points were used, which had a correlation coefficient of 0.999 or higher in a relative pressure range of 0 to 0.2 and exhibited as high correlation as possible.

### 5. Specific Surface Area (Sh₂O) by Water Vapor Adsorption

100 mg of a sample was degassed at 150° C. for 8 hours. A water vapor adsorption isotherm was then measured at constant volume at an adsorption temperature of 298 K using BELSORP-aqua3 (manufactured by BEL JAPAN INC.), and the specific surface area was calculated by a multi-point BET method.

In this multi-point BET method, at least three points were used, which had a correlation coefficient of 0.999 or higher in a relative pressure range of 0 to 0.2 and exhibited as high correlation as possible.

### 6. X-Ray Diffraction Pattern

The X-ray diffraction pattern was measured by irradiation with copper Kα X-rays under the following conditions using an X-ray diffractometer (trade name: RINT2500V, manufactured by Rigaku Corp.).

Output: 40 kV-300 mA
Scan range: 2θ=5 to 30°
Scan speed: 1°/min.
Divergence slit: 1°
Scattering slit: 1°
Receiving slit: 0.3 mm
Sampling width: 0.02°

Interplanar spacing d and peak intensity were calculated under the following set conditions using X-ray diffraction analysis software JADE6 manufactured by MDI (Material Data Incorporated).

Smoothing: smoothing score=15
Background removal: peak width threshold: 0.100°, intensity threshold: 0.01 cps
Kα2 removal: intensity ratio (Kα2/Kα1)=0.50
Peak search: peak width threshold=0.500°, peak intensity threshold=500 cps

### 7. Composition of Reaction Product

The composition was measured using a gas chromatograph (trade name: HP5890 series II, manufactured by Agilent Technologies).

In this context, titanosilicates (I) obtained in production examples below are respectively referred to as catalysts A to M.

Preparation of Catalyst A

In an autoclave, 899 g of piperidine (manufactured by Wako Pure Chemical Industries, Ltd.), 2402 g of pure water, 112 g of tetra-n-butyl orthotitanate [TBOT] (manufactured by Wako Pure Chemical Industries, Ltd.), 565 g of boric acid (manufactured by Wako Pure Chemical Industries, Ltd.), and 410 g of fumed silica (trade name: Cab-O-Sil M7D, manufactured by Cabot Corp.) were dissolved at 25° C. in an air atmosphere and then aged for 1.5 hours. Furthermore, the autoclave was tightly closed, and the obtained gel was heated over 8 hours with stirring and then kept at 160° C. for 96 hours for hydrothermal synthesis to obtain a suspended solution.

After filtration of the obtained suspended solution, the obtained solid matter was washed with water until the pH of the wash was around 10. Next, the solid matter was dried at 50° C. until no decrease in weight was seen, to obtain 522 g of a laminar compound 1.

To 75 g of the laminar compound 1, 3750 mL of 2 M nitric acid was added, and the mixture was refluxed for 20 hours. After filtration of the obtained reaction mixture, the obtained solid matter was washed with water until the pH of the wash was around neutral. The solid matter was then vacuum-dried at 150° C. until no decrease in weight was seen, to obtain 60 g of a white powder (solid product 1). As a result of measuring an X-ray diffraction pattern, the solid product 1 was confirmed to have an MWW precursor structure. The solid product 1 had a Ti content of 1.67% by mass and an Si/N ratio of 105. As a result of measuring an UV-visible absorption spectrum, the solid product 1 was demonstrated to be titanosilicate. The solid product 1 had an SH₂O/SN₂ ratio of 0.58.

Twenty (20) g of the solid product 1 was calcined at 530° C. for 6 hours to obtain 18 g of Ti-MWW (solid product 2). The obtained powder was confirmed by X-ray diffraction pattern measurement to have an MWW structure. The solid product 2 had a Ti content of 1.89% by mass and an Si/N ratio of 2005. As a result of measuring an UV-visible absorption spectrum, the solid product 2 was demonstrated to be titanosilicate. The solid product 1 had an SH₂O/SN₂ ratio of 0.38.

In an autoclave, 200 g of piperidine (manufactured by Wako Pure Chemical Industries, Ltd.), 400 g of pure water, and 135 g of the solid product 2 were dissolved at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was dried in vacuum at 150° C. until no decrease in weight was seen, to obtain 134 g of a white powder (catalyst A).

As a result of measuring an X-ray diffraction pattern, the catalyst A was confirmed to have an MWW precursor structure. The catalyst A had a Ti content of 1.76% by mass and an Si/N ratio of 11. As a result of measuring an UV-visible absorption spectrum, the catalyst A was demonstrated to be titanosilicate. The catalyst A had an SH₂O/SN₂ ratio of 0.99.

Twenty (20) g of the catalyst A was calcined at 530° C. for 6 hours to obtain 18 g of a Ti-MWW powder (solid product 3). The solid product 3 was confirmed by X-ray diffraction pattern measurement to have an MWW structure. The solid product 3 had a Ti content of 1.95% by mass and an Si/N ratio of 1003. As a result of measuring an UV-visible absorption spectrum, the solid product 3 was demonstrated to be titanosilicate. The solid product 3 had an SH₂O/SN₂ ratio of 0.41. On the other hand, to 15 g of the catalyst A, 777 g of 2 N nitric acid was added, and the mixture was refluxed for 20 hours. After filtration of the obtained reaction mixture, the obtained solid matter was washed with water until the pH of the wash was around neutral. The solid matter was vacuum-dried at 150° C. until no decrease in weight was seen, to obtain 12 g of a white powder (solid product 4). As a result of measuring an X-ray diffraction pattern, the solid product 4 was confirmed to have an MWW precursor structure. The solid product 4 had a Ti content of 1.42% by mass and an Si/N ratio of 79. As a result of measuring an UV-visible absorption spectrum, the solid product 4 was demonstrated to be titanosilicate. The solid product 4 had an SH₂O/SN₂ ratio of 0.52.

Preparation of Catalyst B

In an autoclave, 899 g of piperidine (manufactured by Wako Pure Chemical Industries, Ltd.), 2402 g of pure water, 565 g of boric acid (manufactured by Wako Pure Chemical Industries, Ltd.), and 410 g of fumed silica (trade name: Cab-O-Sil M7D, manufactured by Cabot Corp.) were dissolved at 25° C. in an air atmosphere and then aged for 1.5 hours. Furthermore, the autoclave was tightly closed, and the obtained gel was heated over 8 hours with stirring and then kept at 160° C. for 120 hours to obtain a suspended solution. After filtration of the suspended solution, the obtained solid matter was washed with water until the pH of the wash was around 10. Next, the solid matter was dried at 50° C. until no decrease in weight was seen, to obtain 495 g of a white powder b1. As a result of measuring an X-ray diffraction pattern, the white powder b1 was confirmed to have a laminar structure. The white powder b1 had a boron content of 1.5% by weight and a silicon content of 34.8%.

To 75 g of the laminar borosilicate (white powder b1) thus obtained, 3885 g of 2 N nitric acid and 9.5 g of tetra-n-butyl orthotitanate [TBOT] (manufactured by Wako Pure Chemical Industries, Ltd.) were added, and the mixture was refluxed for 20 hours. After filtration of the obtained reaction mixture, the obtained solid matter was washed with water until the pH of the wash was around neutral. The solid matter was vacuum-dried at 150° C. until no decrease in weight was seen, to obtain 60 g of a white powder b2. As a result of measuring an X-ray diffraction pattern, this white powder b2 was confirmed to have an MWW precursor structure. The white powder b2 was demonstrated to have a Ti content of 1.39% by mass and an Si/N ratio of 56. As a result of measuring an UV-visible absorption spectrum, the white powder b2 was demonstrated to be titanosilicate.

Thirty (30) g of the white powder b2 was calcined at 530° C. for 6 hours to obtain 27 g of a powder b3. The obtained powder b3 was confirmed by X-ray diffraction pattern measurement to have an MWW structure. Moreover, the powder b3 had a titanium content of 1.42% by weight measured by ICP emission spectroscopy.

In an autoclave, 40 g of piperidine (manufactured by Wako Pure Chemical Industries, Ltd.), 80 g of pure water, and 27 g of the powder b3 were dissolved at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 26 g of a white powder b4 (catalyst B). As a result of measuring an X-ray diffraction pattern, this white powder b4 was confirmed to have an MWW precursor structure. The catalyst B had a Ti content of 1.40% by mass and an Si/N ratio of 10. As a result of measuring an UV-visible absorption spectrum, the catalyst B was demonstrated to be titanosilicate. The catalyst B had an SH₂O/SN₂ ratio of 1.28.

Preparation of Catalyst C

Forty (40) g of hexamethyleneimine (manufactured by Wako Pure Chemical Industries, Ltd.), 80 g of pure water, and 27 g of the solid product 2 were dissolved in an autoclave at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 26 g of a white powder (catalyst C). As a result of measuring an X-ray diffraction pattern, the catalyst C was confirmed to have an MWW precursor structure. The catalyst C had a Ti content of 1.70% by mass and an Si/N ratio of 12. As a result of measuring an UV-visible absorption spectrum, the catalyst C was demonstrated to be titanosilicate. The catalyst C had an SH₂O/SN₂ ratio of 0.76.

Preparation of Catalyst D

Forty (40) g of piperidine (manufactured by Wako Pure Chemical Industries, Ltd.), 80 g of pure water, and 15 g of the solid product 1 were dissolved in an autoclave at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 11 g of a white powder (catalyst D). As a result of measuring an X-ray diffraction pattern, this white powder was confirmed to have an MWW precursor structure. The catalyst D had a Ti content of 1.78% by mass and an Si/N ratio of 11. As a result of measuring an UV-visible absorption spectrum, the catalyst D was demonstrated to be titanosilicate. The catalyst D had an SH₂O/SN₂ ratio of 0.96.

Preparation of Catalyst E

Sixty (60) g of piperidine (manufactured by Wako Pure Chemical Industries, Ltd.) and 5 g of the solid product 1 were mixed in a glass beaker at 25° C. in an air atmosphere and left standing at 25° C. for 24 hours. Next, after filtration of the suspended solution, the obtained solid matter was washed with water until the pH of the wash was around 9. The solid matter was further vacuum-dried at 150° C. until no decrease in weight was seen, to obtain 4.9 g of a white powder e (catalyst E). As a result of measuring an X-ray diffraction pattern and an UV-visible absorption spectrum, this white powder e was confirmed to have a Ti-MWW precursor structure. The catalyst E had a Ti content of 1.83% by mass and an Si/N ratio of 16.

Preparation of Catalyst F

In an autoclave, 899 g of piperidine (manufactured by Wako Pure Chemical Industries, Ltd.), 2402 g of pure water, 112 g of tetra-n-butyl orthotitanate [TBOT] (manufactured by Wako Pure Chemical Industries, Ltd.), 565 g of boric acid (manufactured by Wako Pure Chemical Industries, Ltd.), and 410 g of fumed silica (trade name: Cab-O-Sil M7D, manufactured by Cabot Corp.) were dissolved at 25° C. in an air atmosphere and then aged for 1.5 hours. Furthermore, the autoclave was tightly closed, and the obtained gel was heated over 8 hours with stirring and then kept at 160° C. for 96 hours for hydrothermal synthesis to obtain a suspended solution. After filtration of the obtained suspended solution, the obtained solid matter was washed with water until the pH of the wash was 10.7. Next, the solid matter was dried at 50° C. until no decrease in weight was seen, to obtain 547 g of a laminar compound.

To 75 g of the laminar compound, 3750 mL of 2 M nitric acid was added, and the mixture was refluxed for 20 hours. After filtration of the obtained reaction mixture, the obtained solid matter was washed with water until the pH of the wash was around neutral. The solid matter was vacuum-dried at 150° C. for 4 hours to obtain 60 g of a white powder f1. As a result of measuring an X-ray diffraction pattern and an UV-visible absorption spectrum, this white powder f1 was confirmed to be a Ti-MWW precursor. As a result of elementary analysis, the white powder f1 had 1.60% by weight of Ti (titanium) and an SUN ratio of 105.

Twenty (20) g of the white powder f1 was calcined at 530° C. for 6 hours to obtain 18 g of Ti-MWW (powder f2). As a result of measuring an X-ray diffraction pattern and an UV-visible absorption spectrum, the powder f2 was confirmed to be Ti-MWW.

Twenty (20) g of piperidine (manufactured by Wako Pure Chemical Industries, Ltd.), 20 g of hexamethyleneimine (manufactured by Wako Pure Chemical Industries, Ltd.), 80 g of pure water, and 10 g of the powder f2 were dissolved in an autoclave at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 10 g of a white powder f3 (catalyst F). As a result of measuring an X-ray diffraction pattern and an UV-visible absorption spectrum, this white powder 13 was confirmed to be titanosilicate. The white powder f3 had a Ti-MWW precursor structure. The catalyst F had a Ti content of 1.65% by mass and an Si/N ratio of 11.

Preparation of Catalyst G

A catalyst G was prepared as follows based on a method described in Chemical Communication 1026-1027, (2002).

In an autoclave, 899 g of piperidine (manufactured by Wako Pure Chemical Industries, Ltd.), 2402 g of pure water, 565 g of boric acid (manufactured by Wako Pure Chemical Industries, Ltd.), and 410 g of fumed silica (trade name: Cab-O-Sil M7D, manufactured by Cabot Corp.) were dissolved at 25° C. in an air atmosphere and then aged for 1.5 hours. Furthermore, the autoclave was tightly closed, and the obtained gel was heated over 8 hours with stirring and then kept at 160° C. for 120 hours for hydrothermal synthesis to obtain a suspended solution. After filtration of the obtained suspended solution, the obtained solid matter was washed with water until the pH of the wash was 10.6. Next, the solid matter was dried at 50° C. until no decrease in weight was seen, to obtain 495 g of a solid product g1 (laminar borosilicate). The solid product g1 had a B content of 1.50% by mass and an Si content of 34.8% by mass.

To 75 g of the solid product g1, 3750 mL of 2 M nitric acid was added, and the mixture was refluxed for 20 hours. After filtration of the obtained reaction mixture, the obtained solid matter was washed with water until the pH of the wash was around neutral. The solid matter was vacuum-dried at 150° C. until no decrease in weight was seen, to obtain 57 g of a white powder g2. As a result of measuring an X-ray diffraction pattern, this white powder g2 was confirmed to have an MWW precursor structure. Forty (40) g of the white powder g2 was calcined at 530° C. for 6 hours to obtain 36 g of a solid product g3 (B-MWW). The solid product g3 was confirmed by X-ray diffraction pattern measurement to have an MWW structure.

In an autoclave, 29 g of piperidine (manufactured by Wako Pure Chemical Industries, Ltd.), 118 g of pure water, 5.3 g of TBOT (manufactured by Wako Pure Chemical Industries, Ltd.), and 20 g of the B-MWW were dissolved at 25° C. in an air atmosphere and then aged for 1.5 hours. Furthermore, the autoclave was tightly closed, and the obtained gel was heated over 8 hours with stirring and then kept at 160° C. for 120 hours for hydrothermal synthesis to obtain a suspended solution. After filtration of the obtained suspended solution, the obtained solid matter was washed with water until the pH of the wash was 10.3. Next, the solid matter was dried at 50° C. until no decrease in weight was seen, to obtain 23 g of a solid product g4.

To 15 g of the solid product g4, 750 mL of 2 M nitric acid was added, and the mixture was refluxed for 20 hours. After filtration of the obtained reaction mixture, the obtained solid matter was washed with water until the pH of the wash was around neutral. The solid matter was vacuum-dried at 150° C. until no decrease in weight was seen, to obtain 12 g of a white powder g5. As a result of measuring an X-ray diffraction pattern and an UV-visible absorption spectrum, this white powder g5 was confirmed to have an Ti-MWW precursor structure. The white powder g5 had a Ti content of 1.94% by mass and an Si/N ratio of 102.

Ten (10) g of the white powder g5 was calcined at 530° C. for 6 hours to obtain 9 g of a solid product g6 (Ti-MWW). The solid product g6 was confirmed by X-ray diffraction pattern measurement to have an MWW structure.

Forty (40) g of piperidine (manufactured by Wako Pure Chemical Industries, Ltd.), 80 g of pure water, and 7 g of the solid product g6 were dissolved in an autoclave at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 6 g of a white powder g7 (catalyst G).

As a result of measuring an X-ray diffraction pattern and an UV-visible absorption spectrum, the white powder g7 was confirmed to be titanosilicate having a Ti-MWW precursor structure. The catalyst G had a Ti content of 1.96% by mass and an Si/N ratio of 13.

Preparation of Catalyst H

In an autoclave, 257 g of piperidine (manufactured by Wako Pure Chemical Industries, Ltd.), 686 g of pure water, 6.4 g of TBOT (manufactured by Wako Pure Chemical Industries, Ltd.), 162 g of boric acid (manufactured by Wako Pure Chemical Industries, Ltd.), and 117 g of fumed silica (trade name: Cab-O-Sil M7D, manufactured by Cabot Corp.) were dissolved at 25° C. in an air atmosphere and then aged for 1.5 hours. Furthermore, the autoclave was tightly closed, and the obtained gel was heated over 8 hours with stirring and then kept at 160° C. for 120 hours for hydrothermal synthesis to obtain a suspended solution. After filtration of the obtained suspended solution, the obtained solid matter was washed with water until the pH of the wash was 10.2.

Next, the solid matter was dried at 50° C. until no decrease in weight was seen, to obtain 125 g of a solid product h1.

To 75 g of the solid product h1, 3750 mL of 2 M nitric acid and 9.5 g of TBOT were added, and the mixture was refluxed for 20 hours. After filtration of the obtained reaction mixture, the obtained solid matter was washed with water until the pH of the wash was around neutral. The solid matter was vacuum-dried at 150° C. until no decrease in weight was seen, to obtain 59 g of a white powder h2. As a result of measuring an X-ray diffraction pattern and an UV-visible absorption spectrum, this white powder h2 was confirmed to be a Ti-MWW precursor. The white powder h2 had a Ti content of 1.67% by mass and an Si/N ratio of 46.

Twenty (20) g of the white powder h2 was calcined at 530° C. for 6 hours to obtain 18 g of a solid product h3 (Ti-MWW). The solid product h3 was confirmed by X-ray diffraction pattern measurement to have an MWW structure.

Forty (40) g of piperidine (manufactured by Wako Pure Chemical Industries, Ltd.), 80 g of pure water, and 10 g of the solid product h3 were dissolved in an autoclave at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 11 g of a white powder h4 (catalyst H). As a result of measuring an X-ray diffraction pattern and an UV-visible absorption spectrum, this white powder h4 was confirmed to be titanosilicate having a Ti-MWW precursor structure. The catalyst H had a Ti content of 1.76% by mass and an Si/N ratio of 10.

Preparation of Catalyst I

In an autoclave, 257 g of piperidine (manufactured by Wako Pure Chemical Industries, Ltd.), 686 g of pure water, 13.2 g of TBOT (manufactured by Wako Pure Chemical Industries, Ltd.), 162 g of boric acid, and 117 g of fumed silica (trade name: Cab-O-Sil M7D) were dissolved at 25° C. in an air atmosphere and then aged for 1.5 hours. Furthermore, the autoclave was tightly closed, and the obtained gel was heated over 8 hours with stirring and then kept at 160° C. for 120 hours for hydrothermal synthesis to obtain a suspended solution. After filtration of the obtained suspended solution, the obtained solid matter was washed with water until the pH of the wash was 10.4. Next, the solid matter was dried at 50° C. until no decrease in weight was seen, to obtain 145 g of a solid product i1.

To 75 g of the solid product i1, 3750 mL of 2 M nitric acid and 9.5 g of TBOT were added, and the mixture was refluxed for 20 hours. After filtration of the obtained reaction mixture, the obtained solid matter was washed with water until the pH of the wash was around neutral. The solid matter was vacuum-dried at 150° C. until no decrease in weight was seen, to obtain 49 g of a white powder i2. As a result of measuring an X-ray diffraction pattern and an UV-visible absorption spectrum, this white powder i2 was confirmed to have a Ti-MWW precursor structure. The white powder i2 was confirmed to have a Ti content of 1.93% by mass and an Si/N ratio of 61.

Thirty (30) g of the white powder i2 was calcined at 530° C. for 6 hours to obtain 27 g of a solid product i3 (Ti-MWW). The obtained solid product i3 was confirmed by X-ray diffraction pattern measurement to have an MWW structure.

Forty (40) g of piperidine (manufactured by Wako Pure Chemical Industries, Ltd.), 80 g of pure water, and 20 g of the solid product i3 were dissolved in an autoclave at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 19 g of a white powder i4 (catalyst I). As a result of measuring an X-ray diffraction pattern and an UV-visible absorption spectrum, the catalyst I was confirmed to be titanosilicate having a Ti-MWW precursor structure. The catalyst I had a Ti content of 2.03% by mass and an Si/N ratio of 11.

Preparation of Catalyst J

In an autoclave, 899 g of piperidine (manufactured by Wako Pure Chemical Industries, Ltd.), 2402 g of pure water, 22.4 g of TBOT (manufactured by Wako Pure Chemical Industries, Ltd.), 565 g of boric acid (manufactured by Wako Pure Chemical Industries, Ltd.), and 410 g of fumed silica (trade name: Cab-O-Sil M7D) were dissolved at 25° C. in an air atmosphere and then aged for 1.5 hours. Furthermore, the autoclave was tightly closed, and the obtained gel was heated over 8 hours with stirring and then kept at 160° C. for 120 hours for hydrothermal synthesis to obtain a suspended solution. After filtration of the obtained suspended solution, the obtained solid matter was washed with water until the pH of the wash was 10.4. Next, the solid matter was dried at 50° C. until no decrease in weight was seen, to obtain 564 g of a solid product j1.

To 75 g of the solid product j1, 3750 mL of 2 M nitric acid and 9.5 g of TBOT were added, and the mixture was refluxed for 20 hours. After filtration of the obtained reaction mixture, the obtained solid matter was washed with water until the pH of the wash was around neutral. The solid matter was further vacuum-dried at 150° C. until no decrease in weight was seen, to obtain 62 g of a white powder j2. As a result of measuring an X-ray diffraction pattern and an UV-visible absorption spectrum, the white powder j2 was confirmed to be a Ti-MWW precursor. The white powder j2 had a Ti content of 1.56% by mass and an Si/N ratio of 55.

60 g of the white powder j2 was calcined at 530° C. for 6 hours to obtain 54 g of a solid product j3 (Ti-MWW). The obtained solid product j3 was confirmed by X-ray diffraction pattern measurement to have an MWW structure. The same procedure as above was further performed twice to obtain a total of 162 g of the solid product j3.

In an autoclave 300 g of piperidine (manufactured by Wako Pure Chemical Industries, Ltd.), 600 g of pure water, and 110 g of the solid product j3 were dissolved at 25° C. in an air atmosphere and then aged for 1.5 hours. Furthermore, the autoclave was tightly closed, and the obtained gel was heated over 4 hours with stirring and then kept at 160° C. for 24 hours to obtain a suspended solution. After filtration of the obtained suspended solution, the obtained solid matter was washed with water until the pH of the wash was around 9. Next, the solid matter was vacuum-dried at 150° C. until no decrease in weight was seen, to obtain 108 g of a white powder j4 (catalyst J). As a result of measuring an X-ray diffraction pattern and an UV-visible absorption spectrum, this white powder j4 was confirmed to be titanosilicate having a Ti-MWW precursor structure. The catalyst J had a Ti content of 1.58% by mass and an Si/N ratio of 10.

Preparation of Catalyst K

The catalyst J was silylated based on a method described in Japanese Patent Laid-Open No. 2003-326171. Specifically, 11 g of 1,1,1,3,3,3-hexamethyldisilazane (manufactured by Wako Pure Chemical Industries, Ltd.), 175 mL of toluene (manufactured by Wako Pure Chemical Industries, Ltd.), and 15 g of the catalyst J were mixed, and the mixture was refluxed for 3 hours for silylation. After filtration of the obtained reaction mixture, the obtained solid matter was washed with 500 mL of acetone and 1 L of a water/acetonitrile (=1/4, weight ratio) mixed solvent in this order and then vacuum-dried at 150° C. until no decrease in weight was seen, to obtain 14 g of a white powder (catalyst K). The catalyst K had a Ti content of 1.61% by mass and an Si/N ratio of 13.

Preparation of Catalyst L

In an autoclave, 899 g of piperidine (manufactured by Wako Pure Chemical Industries, Ltd.), 2402 g of pure water, 112 g of tetra-n-butyl orthotitanate [TBOT] (manufactured by Mitsubishi Gas Chemical Co., Inc.), 565 g of boric acid (manufactured by Wako Pure Chemical Industries, Ltd.), and 410 g of fumed silica (trade name: Cab-O-Sil M7D, manufactured by Cabot Corp.) were dissolved at 25° C. in an air atmosphere and then aged for 1.5 hours. Furthermore, the autoclave was tightly closed, and the obtained gel was heated over 8 hours with stirring and then kept at 160° C. for 120 hours for hydrothermal synthesis to obtain a suspended solution. After filtration of the obtained suspended solution, the obtained solid matter was washed with water until the pH of the wash was 10.8. Next, the solid matter was dried at 50° C. until no decrease in weight was seen, to obtain 518 g of a laminar compound.

To 75 g of the laminar compound, 3750 mL of 2 M nitric acid was added, and the mixture was refluxed for 20 hours. After filtration of the obtained reaction mixture, the obtained solid matter was washed with water until the pH of the wash was around neutral. The solid matter was vacuum-dried at 150° C. for 4 hours to obtain 60 g of a white powder n1. As a result of measuring an X-ray diffraction pattern and an UV-visible absorption spectrum, this white powder n1 was confirmed to be a Ti-MWW precursor. As a result of elementary analysis, the white powder n1 had 1.60% by weight of Ti (titanium) and an Si/N ratio of 90.

Twenty (20) g of the white powder n1 was calcined at 530° C. for 6 hours to obtain 18 g of Ti-MWW (powder n2). As a result of measuring an X-ray diffraction pattern and an UV-visible absorption spectrum, the powder n2 was confirmed to be Ti-MWW.

In an autoclave, 45 g of piperidine (manufactured by Wako Pure Chemical Industries, Ltd.), 90 g of pure water, and 15 g of the powder n2 were dissolved at 25° C. in an air atmosphere and then aged for 0.5 hours. Furthermore, the autoclave was tightly closed, and the obtained gel was heated over 4 hours with stirring and then kept at 160° C. for 16 hours to obtain a suspended solution. After filtration of the obtained suspended solution, the obtained solid matter was vacuum-dried at 50° C. until no decrease in weight was seen, to obtain 5 g of a white powder n3 (catalyst L). As a result of measuring an X-ray diffraction pattern and an UV-visible absorption spectrum, this white powder n3 was confirmed to be titanosilicate having a Ti-MWW precursor structure. The catalyst L had a Ti content of 1.37% by mass and an Si/N ratio of 8.7.

Preparation of Catalyst M

Five (5) g of the catalyst A, 90 g of pure water, and 10 g of acetic acid (manufactured by Wako Pure Chemical Industries, Ltd.) were added to a three-neck glass flask at 25° C. in an air atmosphere, and the mixture was stirred in air at 75° C. for 6 hours. After filtration of the obtained suspended solution, the obtained solid matter was washed with water until the pH of the wash was 6.7. Next, the solid matter was dried at 150° C. until no decrease in weight was seen, to obtain 3.9 g of a white powder m1 (catalyst M). As a result measuring an X-ray diffraction pattern and an UV-visible absorption spectrum, this white powder m1 was confirmed to be titanosilicate having a Ti-MWW precursor structure. The catalyst M had a Ti content of 1.82% by mass and an Si/N ratio of 31.

Pd/Active Carbon (AC) Catalyst

A Pd/active carbon (AC) catalyst was prepared by the following method. To a 1-L eggplant-shaped flask, 3 g of active carbon (manufactured by Wako Pure Chemical Industries, Ltd.) washed in advance with 2 L of water, and 300 mL of water were added, and the mixture was stirred in air at 25° C. To this suspension, 40 mL of an aqueous solution containing 0.3 mmol of Pd tetraammine chloride separately prepared was gradually added dropwise in air at 25° C. After the completion of the dropwise addition, the suspension was further stirred in air at 25° C. for 6 hours. After the completion of the stirring, the moisture was removed using a rotary evaporator, and the residue was vacuum-dried at 80° C. for 6 hours and further calcined in a nitrogen atmosphere at 300° C. for 6 hours to obtain a Pd/AC catalyst.

Tables 1 to 4 show X-ray diffraction pattern data on the catalysts A to M, the solid products 1 to 5, the powders b3 and f2, the solid products g6, h3, and i3, and the powder n2.

TABLE 1
Interplanar spacing d [Å]

| | Catalyst | | | | | |
|---|---|---|---|---|---|---|
| | Catalyst | | Catalyst | Catalyst | Catalyst | Catalyst |
| | A | Catalyst B | C | D | E | F |
| 12.4 ± 0.8 | 12.05 | 12.05 | 12.15 | 11.98 | 12.18 | 11.86 |
| 10.8 ± 0.3 | 10.87 | 10.84 | 10.92 | 10.84 | 10.92 | 10.68 |
| 9.0 ± 0.3 | 8.83 | 8.81 | 8.85 | 8.81 | 8.86 | 8.71 |
| 6.0 ± 0.3 | 6.07 | 6.06 | 6.09 | 6.07 | 6.09 | 6.02 |
| 3.9 ± 0.1 | 3.88 | 3.88 | 3.89 | 3.88 | 3.89 | 3.86 |
| 3.4 ± 0.1 | 3.38 | 3.38 | 3.39 | 3.38 | 3.39 | 3.37 |
| Intensity | 0.16 | 0.15 | 0.15 | 0.13 | 0.22 | 0.15 |
| ratio | | | | | | |
| X¹/X² | | | | | | |

In each table, X¹/X² represents the ratio of peak intensity X¹ at the interplanar spacing 9.0±0.3 Å to peak intensity X² at the interplanar spacing 3.4±0.1 Å.

TABLE 2
Interplanar spacing d [Å]

| | Catalyst | | | | | |
|---|---|---|---|---|---|---|
| | Catalyst | | Catalyst | Catalyst | Catalyst | Catalyst |
| | G | Catalyst H | I | J | K | L |
| 12.4 ± 0.8 | 11.98 | 12.32 | 12.12 | 12.05 | 11.98 | 11.92 |
| 10.8 ± 0.3 | 10.79 | 11.08 | 10.92 | 10.87 | 10.81 | 10.76 |
| 9.0 ± 0.3 | 8.71 | 8.99 | 8.83 | 8.86 | 8.81 | 8.79 |
| 6.0 ± 0.3 | 6.06 | 6.14 | 6.10 | 6.07 | 6.05 | 6.03 |
| 3.9 ± 0.1 | 3.87 | 3.91 | 3.89 | 3.88 | 3.87 | 3.88 |
| 3.4 ± 0.1 | 3.38 | 3.40 | 3.39 | 3.38 | 3.37 | 3.38 |
| X¹/X² | 0.19 | 0.13 | 0.14 | 0.10 | 0.15 | 0.07 |

TABLE 3
Interplanar spacing d [Å]

| | | | Solid | Solid | Solid | |
|---|---|---|---|---|---|---|
| | Catalyst | Solid | product | product | product | Powder |
| Catalyst | M | product 1 | 2 | 3 | 4 | b3 |
| 12.4 ± 0.8 | 12.39 | 12.08 | 11.98 | 12.12 | 11.76 | 12.15 |
| 10.8 ± 0.3 | 11.08 | 10.81 | 10.73 | 10.89 | 10.56 | 10.87 |
| 9.0 ± 0.3 | 8.90 | 8.85 | 8.76 | 8.78 | 8.59 | 8.79 |
| 6.0 ± 0.3 | 6.15 | 6.07 | 6.05 | 6.09 | 5.99 | 6.08 |
| 3.9 ± 0.1 | 3.91 | 3.88 | 3.87 | 3.89 | 3.85 | 3.89 |
| 3.4 ± 0.1 | 3.41 | 3.38 | 3.38 | 3.39 | 3.36 | 3.39 |
| X¹/X² | 0.39 | 0.57 | 0.75 | 0.65 | 0.70 | 0.76 |

TABLE 4
Interplanar spacing d [Å]

| | | Solid | Solid | Solid | Solid | |
|---|---|---|---|---|---|---|
| | Powder | product | product | product | product | Powder |
| Catalyst | f2 | g6 | h3 | i3 | j3 | n2 |
| 12.4 ± 0.8 | 11.95 | 12.28 | 12.02 | 12.12 | 12.14 | 12.08 |
| 10.8 ± 0.3 | 10.71 | 10.97 | 10.79 | 10.89 | 10.87 | 10.81 |
| 9.0 ± 0.3 | 8.72 | 8.85 | 8.72 | 8.79 | 8.83 | 8.79 |
| 6.0 ± 0.3 | 6.05 | 6.14 | 6.07 | 6.10 | 6.09 | 6.08 |
| 3.9 ± 0.1 | 3.88 | 3.91 | 3.88 | 3.89 | 3.89 | 3.89 |
| 3.4 ± 0.1 | 3.38 | 3.41 | 3.38 | 3.39 | 3.39 | 3.39 |
| X¹/X² | 0.74 | 0.53 | 0.70 | 0.65 | 0.67 | 0.67 |

All the catalysts, when used in Examples 1 to 4 and Comparative Examples 1 to 4 below, were contacted with hydrogen peroxide according to the following method prior to reaction. The catalyst was placed at a temperature of 25° C. for 1 hour in a water/acetonitrile (=1/4 (weight ratio)) solution containing 0.1% by weight of hydrogen peroxide at a proportion of 100 g of the solution to 0.05 g of the catalyst. After filtration of the solution containing the catalyst, the collected catalyst was washed with 500 mL of water. The catalyst thus washed was further vacuum-dried at 150° C. for 1 hour and then subjected to the reaction.

### Example 1

A 30% aqueous H₂O₂ solution (manufactured by Wako Pure Chemical Industries, Ltd.), acetonitrile, and ion-exchanged water were used to prepare a solution of H₂O₂: 0.2% by weight, water: 19.96% by weight, and acetonitrile: 79.84% by weight. Sixty (60) g of the prepared solution and 0.010 g of the catalyst A treated in advance with hydrogen peroxide were charged into a 100-mL stainless autoclave. Next, the autoclave was transferred into an ice bath, and 1.2 g of liquid propylene was charged thereinto. The pressure within the reaction system was further increased to 2 MPa-G using argon. The autoclave was placed in a hot-water bath at 60° C. and taken out of the hot-water bath after 1 hour. Sampling was performed, and the sample was analyzed using a gas chromatograph. As a result, propylene oxide was produced at a yield of 3.86 mmol.

### Example 2

Propylene oxide production was performed by the same procedure as in Example 1 except that the catalyst B was used instead of the catalyst A. As a result, propylene oxide was produced at a yield of 3.40 mmol.

### Example 3

Propylene oxide production was performed by the same procedure as in Example 1 except that the catalyst D was used instead of the catalyst A. As a result, propylene oxide was produced at a yield of 3.73 mmol.

### Example 4

Propylene oxide production was performed by the same procedure as in Example 1 except that the catalyst E was used instead of the catalyst A. As a result, propylene oxide was produced at a yield of 3.73 mmol.

### Comparative Example 1

Propylene oxide production was performed by the same procedure as in Example 1 except that the solid product 1 was used instead of the catalyst A. As a result, propylene oxide was produced at a yield of 3.21 mmol.

### Comparative Example 2

Propylene oxide production was performed by the same procedure as in Example 1 except that the solid product 2 was used instead of the catalyst A. As a result, propylene oxide was produced at a yield of 2.59 mmol.

### Comparative Example 3

Propylene oxide production was performed by the same procedure as in Example 1 except that the solid product 3 was used instead of the catalyst A. As a result, propylene oxide was produced at a yield of 3.18 mmol.

### Comparative Example 4

Propylene oxide production was performed by the same procedure as in Example 1 except that the solid product 4 was used instead of the catalyst A. As a result, propylene oxide was produced at a yield of 2.71 mmol.

### Example 5

Continuous reaction was performed under conditions involving a temperature of 60° C., a pressure of 4 MPa (gage pressure), and a residence time of 15 minutes, in which 1.98 g of the catalyst A was placed in a 0.5-L autoclave, and nitrogen at a rate of 500 mL/min., propylene at a rate of 92 g/Hr, and a water/acetonitrile (weight ratio, water/acetonitrile=20/80) solution of 7% by weight of H₂O₂ at a rate of 652 mL/Hr were supplied thereto while the reaction mixture was extracted via a filter from the autoclave.

Liquid and gas phases extracted after 9 hours into the reaction were respectively analyzed using a gas chromatograph and determined to have propylene oxide produced at a yield of 730 mmol/Hr, propylene glycol produced at a yield of 6.87 mmol/Hr, and a hydrogen peroxide conversion rate of 98.2%.

### Example 6

Propylene oxide production was performed by the same procedure as in Example 5 except that the catalyst B was used instead of the catalyst A. Liquid and gas phases extracted after 32 hours into the reaction were respectively analyzed using a gas chromatograph and determined to have propylene oxide produced at a yield of 737 mmol/Hr, propylene glycol produced at a yield of 7.44 mmol/Hr, and a hydrogen peroxide conversion rate of 98.5%.

### Example 7

Propylene oxide production was performed by the same procedure as in Example 5 except that the catalyst C was used instead of the catalyst A. Liquid and gas phases extracted after 6 hours into the reaction were respectively analyzed using a gas chromatograph and determined to have propylene oxide produced at a yield of 715 mmol/Hr, propylene glycol produced at a yield of 3.25 mmol/Hr, and a hydrogen peroxide conversion rate of 93.7%.

### Example 8

Propylene oxide production was performed by the same procedure as in Example 5 except that the catalyst D was used instead of the catalyst A. Liquid and gas phases extracted after 6 hours into the reaction were respectively analyzed using a gas chromatograph and determined to have propylene oxide produced at a yield of 744 mmol/Hr, propylene glycol produced at a yield of 11.10 mmol/Hr, and a hydrogen peroxide conversion rate of 98.1%.

### Example 9

Propylene oxide production was performed by the same procedure as in Example 5 except that the catalyst E was used instead of the catalyst A. Liquid and gas phases extracted after 6 hours into the reaction were respectively analyzed using a gas chromatograph and determined to have propylene oxide produced at a yield of 677 mmol/Hr, propylene glycol produced at a yield of 5.53 mmol/Hr, and a hydrogen peroxide conversion rate of 89.9%.

### Example 10

Propylene oxide production was performed by the same procedure as in Example 5 except that the catalyst F was used instead of the catalyst A. Liquid and gas phases extracted after 6 hours into the reaction were respectively analyzed using a gas chromatograph and determined to have propylene oxide produced at a yield of 633 mmol/Hr, propylene glycol produced at a yield of 2.36 mmol/Hr, and a hydrogen peroxide conversion rate of 93.8%.

### Example 11

Propylene oxide production was performed by the same procedure as in Example 5 except that the catalyst G was used instead of the catalyst A. Liquid and gas phases extracted after 6 hours into the reaction were respectively analyzed using a gas chromatograph and determined to have propylene oxide produced at a yield of 665 mmol/Hr, propylene glycol produced at a yield of 7.00 mmol/Hr, and a hydrogen peroxide conversion rate of 98.8%.

### Comparative Example 5

Propylene oxide production was performed by the same procedure as in Example 5 except that the solid product 1 was used instead of the catalyst A. Liquid and gas phases extracted after 6 hours into the reaction were respectively analyzed using a gas chromatograph and determined to have propylene oxide produced at a yield of 606 mmol/Hr, propylene glycol produced at a yield of 4.52 mmol/Hr, and a hydrogen peroxide conversion rate of 79.5%.

### Example 12

Continuous reaction was performed under conditions involving a temperature of 60° C., a pressure of 3 MPa (gage pressure), and a residence time of 9 minutes, in which 0.3 g of the catalyst G was placed in a 0.5-L autoclave, and then nitrogen at a rate of 500 mL/min., propylene at a rate of 2162 mmol/Hr, and a water/acetonitrile (weight ratio=water/acetonitrile=20/80) solution of 7% by weight of H₂O₂ at a rate of 633 mL/Hr were supplied thereto while the reaction mixture was extracted via a filter from the autoclave. Liquid and gas phases extracted after 2 hours into the reaction were respectively analyzed using a gas chromatograph and determined to have propylene oxide produced at a yield of 516 mmol/Hr, propylene glycol produced at a yield of 0.72 mmol/Hr, and a hydrogen peroxide conversion rate of 69.8%.

### Example 13

Propylene oxide production was performed by the same procedure as in Example 12 except that the catalyst H was used instead of the catalyst G Liquid and gas phases extracted after 1 hour into the reaction were respectively analyzed using a gas chromatograph and determined to have propylene oxide produced at a yield of 652 mmol/Hr, propylene glycol produced at a yield of 3.96 mmol/Hr, and a hydrogen peroxide conversion rate of 88.3%.

### Example 14

Propylene oxide production was performed by the same procedure as in Example 12 except that the catalyst I was used instead of the catalyst G Liquid and gas phases extracted after 1 hour into the reaction were respectively analyzed using a gas chromatograph and determined to have propylene oxide produced at a yield of 695 mmol/Hr, propylene glycol produced at a yield of 3.79 mmol/Hr, and a hydrogen peroxide conversion rate of 96.4%.

### Example 15

Propylene oxide production was performed by the same procedure as in Example 12 except that the catalyst J was used instead of the catalyst G Liquid and gas phases extracted after 1 hour into the reaction were respectively analyzed using a gas chromatograph and determined to have propylene oxide produced at a yield of 495 mmol/Hr, propylene glycol produced at a yield of 0.87 mmol/Hr, and a hydrogen peroxide conversion rate of 65.7%.

### Example 16

Propylene oxide production was performed by the same procedure as in Example 12 except that the catalyst K was used instead of the catalyst G Liquid and gas phases extracted after 1 hour into the reaction were respectively analyzed using a gas chromatograph and determined to have propylene oxide produced at a yield of 485 mmol/Hr, propylene glycol produced at a yield of 0.51 mmol/Hr, and a hydrogen peroxide conversion rate of 65.2%.

### Example 17

Propylene oxide production was performed by the same procedure as in Example 12 except that the catalyst L was used instead of the catalyst G Liquid and gas phases extracted after 1 hour into the reaction were respectively analyzed using a gas chromatograph and determined to have propylene oxide produced at a yield of 535 mmol/Hr, propylene glycol produced at a yield of 0.48 mmol/Hr, and a hydrogen peroxide conversion rate of 71.8%.

### Example 18

Propylene oxide production was performed by the same procedure as in Example 12 except that the catalyst M was used instead of the catalyst G Liquid and gas phases extracted after 1 hour into the reaction were respectively analyzed using a gas chromatograph and determined to have propylene oxide produced at a yield of 522 mmol/Hr, propylene glycol produced at a yield of 0.56 mmol/Hr, and a hydrogen peroxide conversion rate of 71.5%.

### Comparative Example 6

Propylene oxide production was performed by the same procedure as in Example 12 except that the catalyst M was used instead of the catalyst G Liquid and gas phases extracted after 1 hour into the reaction were respectively analyzed using a gas chromatograph and determined to have propylene oxide produced at a yield of 522 mmol/Hr, propylene glycol produced at a yield of 0.56 mmol/Hr, and a hydrogen peroxide conversion rate of 71.5%.

All the catalysts, when used in Examples 19 to 21 and Comparative Examples 7 to 9 below, were contacted with hydrogen peroxide according to the following method prior to reaction. The catalyst was placed at a temperature of 25° C. for 1 hour in a water/acetonitrile (=1/4, weight ratio) solution containing 0.1% by weight of hydrogen peroxide at a proportion of 100 g of the solution to 0.266 g of the catalyst. After filtration of the solution containing the catalyst, the collected catalyst was washed with 500 mL of water.

### Example 19

Continuous reaction was performed under conditions involving a temperature of 60° C., a pressure of 0.8 MPa (gage pressure), and a residence time of 90 minutes, in which 0.266 g of the catalyst A treated in advance with hydrogen peroxide and 0.03 g of the Pd/AC catalyst were charged into a 0.5-L autoclave, and then a source gas comprising propylene/oxygen/hydrogen/nitrogen at a volume ratio of 4/4/10/82 and a water/acetonitrile (=20/80, weight ratio) solution containing 0.7 mmol/kg of anthraquinone and 1% by weight of propylene oxide were supplied thereto at rates of 16 L/Hr and 108 mL/Hr, respectively while the reaction mixture was extracted via a filter from the autoclave. Liquid and gas phases extracted after 5 hours into the reaction were respectively analyzed using a gas chromatograph and determined to have propylene oxide produced at a yield of 6.60 mmol/Hr and propylene glycol selectivity of 6.5%.

### Example 20

Propylene oxide production was performed by the same procedure as in Example 19 except that the catalyst B was used instead of the catalyst A. Liquid and gas phases extracted after 6 hours into the reaction were respectively analyzed using a gas chromatograph and determined to have propylene oxide produced at a yield of 6.27 mmol/Hr and propylene glycol selectivity of 3.7%.

### Example 21

Propylene oxide production was performed by the same procedure as in Example 19 except that the catalyst D was used instead of the catalyst A. Liquid and gas phases extracted after 6 hours into the reaction were respectively analyzed using a gas chromatograph and determined to have propylene oxide produced at a yield of 7.19 mmol/Hr and propylene glycol selectivity of 9.7%.

### Comparative Example 7

Propylene oxide production was performed by the same procedure as in Example 19 except that the solid product 1 was used instead of the catalyst A. Liquid and gas phases extracted after 6 hours into the reaction were respectively analyzed using a gas chromatograph and determined to have propylene oxide produced at a yield of 5.64 mmol/Hr and propylene glycol selectivity of 9.3%.

### Comparative Example 8

Propylene oxide production was performed by the same procedure as in Example 19 except that the solid product 3 was used instead of the catalyst A. Liquid and gas phases extracted after 6 hours into the reaction were respectively analyzed using a gas chromatograph and determined to have propylene oxide produced at a yield of 5.56 mmol/Hr and propylene glycol selectivity of 10.6%.

### Comparative Example 9

Propylene oxide production was performed by the same procedure as in Example 19 except that the solid product 4 was used instead of the catalyst A. Liquid and gas phases extracted after 6 hours into the reaction were respectively analyzed using a gas chromatograph and determined to have propylene oxide produced at a yield of 3.59 mmol/Hr and propylene glycol selectivity of 8.9%.

All the catalysts, when used in Examples 22 to 25 below, were treated with hydrogen peroxide according to the following method prior to reaction. The catalyst was placed at a temperature of 25° C. for 1 hour in a water/acetonitrile (=1/4, weight ratio) solution containing 0.1% by weight of hydrogen peroxide at a proportion of 100 g of the solution to 0.05 g of the catalyst. After filtration of the solution containing the catalyst, the collected catalyst was washed with 500 mL of water. The catalyst thus washed was further vacuum-dried at 150° C. for 1 hour and then subjected to the reaction.

### Example 22

A 30% aqueous H₂O₂ solution (manufactured by Wako Pure Chemical Industries, Ltd.), acetonitrile, and ion-exchanged water were used to prepare a solution of H₂O₂: 0.5% by weight, water: 19.9% by weight, and acetonitrile: 79.6% by weight. 60 g of the prepared solution and 0.010 g of the catalyst A treated in advance with hydrogen peroxide were charged into a 100-mL stainless autoclave. Next, the autoclave was transferred into an ice bath, and 1.2 g of liquid propylene was charged thereinto. The pressure within the reaction system was further increased to 2 MPa-G using argon. The autoclave was placed in a hot-water bath at 60° C. and taken out of the hot-water bath after 1 hour. Sampling was performed, and the sample was analyzed using a gas chromatograph. Propylene oxide was produced at a yield of 4.51 mmol.

### Example 23

Propylene oxide production was performed by the same procedure as in Example 22 except that benzonitrile was used instead of acetonitrile. Propylene oxide was produced at a yield of 5.66 mmol.

### Example 24

Propylene oxide production was performed by the same procedure as in Example 22 except that t-butanol was used instead of acetonitrile. Propylene oxide was produced at a yield of 8.06 mmol.

### Example 25

Propylene oxide production was performed by the same procedure as in Example 22 except that methanol was used instead of acetonitrile. Propylene oxide was produced at a yield of 2.27 mmol.

### INDUSTRIAL APPLICABILITY

The production method of the present invention can produce an oxidized compound at a high yield with high selectivity and is therefore industrially useful. The titanosilicate (I) is useful as a catalyst in the production method.

What is claimed is:

1. A method for producing an oxidized compound, comprising reacting an organic compound with an oxidizing agent in the presence of titanosilicate (I) or a silylated form thereof, the titanosilicate (I) being obtained by contacting titanosilicate (II) with a structure-directing agent, and the titanosilicate (II) having an X-ray diffraction pattern reproduced in the form of interplanar spacings d of
24±0.08 nm,
1.08±0.03 nm,
0.9±0.03 nm,
6±0.03 nm,
0.39±0.01 nm and
0.34±0.01 nm.

2. The method for producing an oxidized compound according to claim 1, wherein the organic compound is an olefin compound or an aromatic compound.

3. The method for producing an oxidized compound according to claim 1, wherein the titanosilicate (I) has a molar ratio of silicon to nitrogen (Si/N ratio) of from 5 to 20 inclusive.

4. The method for producing an oxidized compound according to claim 1, wherein the titanosilicate (I) has a ratio of a specific surface area (SH₂O) to a specific surface area (SN₂) (SH₂O/SN₂) of from 0.7 to 1.5 inclusive, the specific surface areas SH₂O and SN₂ being measured by water vapor adsorption and nitrogen adsorption methods, respectively.

5. The method for producing an oxidized compound according to claim 1, wherein the titanosilicate (II) is crystalline titanosilicate having an MWW or MSE structure, or a Ti-MWW precursor (a).

6. The method for producing an oxidized compound according to claim 1, wherein the structure-directing agent is piperidine or hexamethyleneimine, or a mixture thereof.

7. The method for producing an oxidized compound according to claim 1, wherein the contact of the titanosilicate (II) with the structure-directing agent is performed at a temperature of 0 to 250° C.

8. Titanosilicate or a silylated form thereof, wherein the titanosilicate has a molar ratio of silicon to nitrogen (SUN ratio) of from 10 to 20 inclusive.

9. The titanosilicate or a silylated form thereof according to claim 8, the titanosilicate being obtained by contacting titanosilicate (II) with a structure-directing agent, and the titanosilicate (II) having an X-ray diffraction pattern reproduced in the form of interplanar spacings d of
24±0.08 nm,
1.08±0.03 nm,
9±0.03 nm,
0.6±0.03 nm,
0.39±0.01 nm and
0.34±0.01 nm.

10. The titanosilicate or a silylated form thereof according to claim 9, wherein the titanosilicate (II) is crystalline titanosilicate having an MWW or MSE structure, or a Ti-MWW precursor (a).

11. Use of titanosilicate or a silylated form thereof according to claim 8 as a catalyst in a method for producing an oxidized compound.

12. A catalyst for oxidation reaction of an organic compound, comprising titanosilicate (I) or a silylated form thereof, the titanosilicate (I) being obtained by contacting titanosilicate (II) with a structure-directing agent, and the titanosilicate (II) having an X-ray diffraction pattern reproduced in the form of interplanar spacings d of
1.24±0.08 nm,
08±0.03 nm,
0.9±0.03 nm,
0.6±0.03 nm,
0.39±0.01 nm and
0.34±0.01 nm.

13. The method for producing an oxidized compound according to claim 1, wherein the oxidizing agent is oxygen or peroxide.

14. The method for producing an oxidized compound according to claim 13, wherein the peroxide is at least one compound selected from the group consisting of hydrogen peroxide, t-butyl hydroperoxide, t-amyl hydroperoxide, cumene hydroperoxide, methylcyclohexyl hydroperoxide, tetralin hydroperoxide, isobutylbenzene hydroperoxide, ethylnaphthalene hydroperoxide, and peracetic acid.

15. The method for producing an oxidized compound according to claim 1, wherein the reaction is epoxidation reaction of an olefin compound or hydroxylation reaction of benzene or a phenol compound.

16. The method for producing an oxidized compound according to claim 1, wherein the reaction is epoxidation reaction of an olefin compound, and the oxidizing agent is hydrogen peroxide.

17. The method for producing an oxidized compound according to claim 16, wherein the oxidizing agent is hydrogen peroxide synthesized in the same reaction system as that of the epoxidation of an olefin compound.

18. The method for producing an oxidized compound according to claim 1, wherein the reaction is performed in the presence of an organic solvent selected from the group consisting of alcohol, ketone, nitrile, ether, aliphatic hydrocarbon, aromatic hydrocarbon, halogenated hydrocarbon, ester and mixtures thereof.

19. The method for producing an oxidized compound according to claim 18, wherein the organic solvent is acetonitrile or t-butanol.
